(19) United States
(12) Patent Application Publication
(10) Pub. No.: US 2021/0118136 A1
(43) Pub. Date: Apr. 22, 2021

(54) ARTIFICIAL INTELLIGENCE FOR PERSONALIZED ONCOLOGY

(71) Applicant: Novateur Research Solutions LLC, Ashburn, VA (US)

(72) Inventors: Khurram HASSAN-SHAFIQUE, Aldie, VA (US); Zeeshan RASHEED, Great Falls, VA (US); Jonathan Jacob AMAZON, Herndon, VA (US); Rashid CHOTANI, Great Falls, VA (US)

(73) Assignee: Novateur Research Solutions LLC, Ashburn, VA (US)

(21) Appl. No.: 17/078,012

(22) Filed: Oct. 22, 2020

Related U.S. Application Data
(60) Provisional application No. 62/924,668, filed on Oct. 22, 2019.

(51) Int. Cl. G06T 7/00; G16H 30/20; G16H 50/20; G16H 10/60; G06F 16/535; G16B 40/00; G16B 50/00

(52) U.S. Cl. CPC G06T 7/0012; G16H 30/20; G16H 50/20; G16H 10/60; G06K 9/00147; G16B 40/00; G16B 50/00; G06T 2207/10056; G06T 2207/20081; G06F 16/535

(57) ABSTRACT

Techniques performed by a data processing system for operating a personalized oncology system herein include accessing a first histopathological image of a histopathological slide of a sample taken from a first patient; analyzing the first histopathological image using a first machine learning model configured to extract first features from the first histopathological image; searching a histological database that includes a plurality of second histopathological images and corresponding clinical data for a plurality of second patients to generate search results; analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and presenting an interactive visual representation of the associated statistics and metrics including information for the personalized therapeutic plan for treating the first patient.

### CROSS-REFERENCE TO RELATED APPLICATION

This application claims the benefit of priority to U.S. Provisional Patent Application No. 62/924,668, filed on Oct. 22, 2019 and entitled “Artificial Intelligence for Personalized Oncology,” the entirety of which is incorporated by reference herein in its entirety.

### BACKGROUND

Histopathology refers to microscopic examination of tissue to study the manifestation of disease. In histopathology, a pathologist examines a biopsy or surgical specimen that has been processed and placed on a slide for examination using a microscope. There are numerous histopathologic data sources which include digitally scanned slides that may be used as a reference for diagnosing oncological problems in patient. However, the sheer size and volume of this data makes it impractical for a pathologist, oncologist, or other doctor treating a patient to manually review these data sources. Some attempts to use machine learning models automate this comparison of these data sources with patient data has been attempted. However, these attempts have met with limit success due to numerous issues, including a lack of annotated training data that may be used to train such models and that most histopathologic data does not lend itself to such machine learning approaches due to the sheer size of most histopathologic slide data. Hence, there is a need for improved systems and methods of analyzing oncology data to provide personalized therapeutic plans for treating patients.

### SUMMARY

An example system for personalized oncology according to the disclosure includes a processor and a memory in communication with the processor. The memory comprising executable instructions that, when executed by the processor, cause the processor to control the system to perform functions of: accessing a first histopathological image of a histopathological slide of a sample taken from a first patient; analyzing the first histopathological image using a first machine learning model configured to extract first features from the first histopathological image, wherein the first features are indicative of cancerous tissue in the sample taken from the first patient; searching a histological database that includes a plurality of second histopathological images and corresponding clinical data for a plurality of second patients to generate search results, wherein the search results include a plurality of third histopathological images and corresponding clinical data from the plurality of second histopathological images and corresponding clinical data that match the first features from the first histopathological image, and wherein the third histopathological images and corresponding clinical data are associated with a plurality of third patients of the plurality of second patients; analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and presenting an interactive visual representation of the associated statistics and metrics on a display of the system.

An example method of operating a personalized oncology system according to the disclosure includes accessing a first histopathological image of a histopathological slide of a sample taken from a first patient; analyzing the first histopathological image using a first machine learning model configured to extract first features from the first histopathological image, wherein the first features are indicative of cancerous tissue in the sample taken from the first patient; searching a histological database that includes a plurality of second histopathological images and corresponding clinical data for a plurality of second patients to generate search results, wherein the search results include a plurality of third histopathological images and corresponding clinical data from the plurality of second histopathological images and corresponding clinical data that match the first features from the first histopathological image, and wherein the third histopathological images and corresponding clinical data are associated with a plurality of third patients of the plurality of second patients; analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and presenting an interactive visual representation of the associated statistics and metrics on a display of the system.

An example non-transitory computer readable medium according to the disclosure contains instructions which, when executed by a processor, cause a computer to perform functions of accessing a first histopathological image of a histopathological slide of a sample taken from a first patient; analyzing the first histopathological image using a first machine learning model configured to extract first features from the first histopathological image, wherein the first features are indicative of cancerous tissue in the sample taken from the first patient; searching a histological database that includes a plurality of second histopathological images and corresponding clinical data for a plurality of second patients to generate search results, wherein the search results include a plurality of third histopathological images and corresponding clinical data from the plurality of second histopathological images and corresponding clinical data that match the first features from the first histopathological image, and wherein the third histopathological images and corresponding clinical data are associated with a plurality of third patients of the plurality of second patients; analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and presenting an interactive visual representation of the associated statistics and metrics on a display of the system.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The drawing figures depict one or more implementations in accord with the present teachings, by way of example only, not by way of limitation. In the figures, like reference numerals refer to the same or similar elements. Furthermore, it should be understood that the drawings are not necessarily to scale.

FIG. 1 is a block diagram showing an example computing environment in which an example personalized oncology system may be implemented.

FIG. 2 illustrates an implementation of a deep convolutional neural architecture for image feature learning and extraction.

FIG. 3 shows high-resolution breast cancer histopathological images labeled by pathologists.

FIG. 4 illustrates broad variability of high-resolution image appearances due to high coherency of cancerous cells, extensive inhomogeneity of color distribution, and inter-class variability.

FIG. 5 shows example images of different grades of breast tumors which demonstrate examples of the wide variety of histological patterns that may be present in histopathological samples.

FIG. 6 shows an implementation of a high-level workflow of the AI-based personalized oncology system.

FIG. 7 shows a query patch being matched to a database slide indexed using patches at two different magnification.

FIGS. 8, 9, and 10 show example user interfaces for searching and presenting search results that may provide a personalized therapeutic plan for a patient.

FIG. 11 is a block diagram showing an example computer system upon which aspects of this disclosure may be implemented.

FIG. 12 is a flow chart of an example process for generating personalized therapeutic plan for a patient requiring oncological treatment.

FIG. 13 is a block diagram that shows an example Siamese network.

FIG. 14 is a block diagram that shows an example of another Siamese network.

FIG. 15 is a block diagram that shows an example of a Generative Adversarial Network.

FIG. 16 is a block diagram that shows an example of a style-transfer network.

FIG. 17 is a diagram of a process for feature computation, dictionary learning, and indexing of the image corpus of the historical database 150.

FIG. 18 is a diagram of an example process for performing a query using the personalized oncology system shown in the preceding figures.

FIG. 19 is a flow chart of another example process for generating personalized therapeutic plan for a patient requiring oncological treatment.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

Techniques provided herein provide technical solutions for the problem of providing an optimized and personalized therapeutic plan for a patient requiring oncological treatment. The techniques disclosed herein utilize artificial intelligence models trained on histological and associated clinical data to infer the efficacy of various treatments for a patient and to provide the patient's oncologist with key insights about clinical outcomes of the treatments, including a survival rate, reoccurrence rate, time-to-reoccurrence, and/or other factors associated with these treatments. The techniques provided herein provide a technical solution to the technical problem of the large amount of annotated training data required by current deep-learning approaches to analyzing image data. The technical solution leverages the knowledge and expertise of trained pathologists to recognize and interpret subtle histologic features and to guide the artificial intelligence system by identifying regions of interest (ROI) in a patient's histopathology imagery to be analyzed. This approach provides the technical benefit of significantly reducing the amount of image data that needs to be analyzed by the deep convolutional neural network.

An artificial intelligence (AI)-based personalized oncology system is provided. The personalized oncology system utilizes a hybrid computer-human system approach that combines (i) the computational power and storage of modern computer systems to mine large histopathological imagery databases, (ii) novel deep learning methods to extract meaningful features from histopathological images without requiring large amounts of annotated training data, (iii) recent advances in large-scale indexing and retrieval of image databases, and (iv) the knowledge and expertise of trained pathologists to recognize and interpret subtle histologic features and to guide the artificial intelligence system. The personalized oncology system exploits histologic imagery and associated clinical data to provide oncologists key insights about clinical outcomes, such as but not limited to survival rate, reoccurrence rate, and time-to-reoccurrence, and the efficacy of treatments based on patient's histological and other personal factors. Thus, the personalized oncology system enables the oncologist to identify optimal treatment plan for the patient.

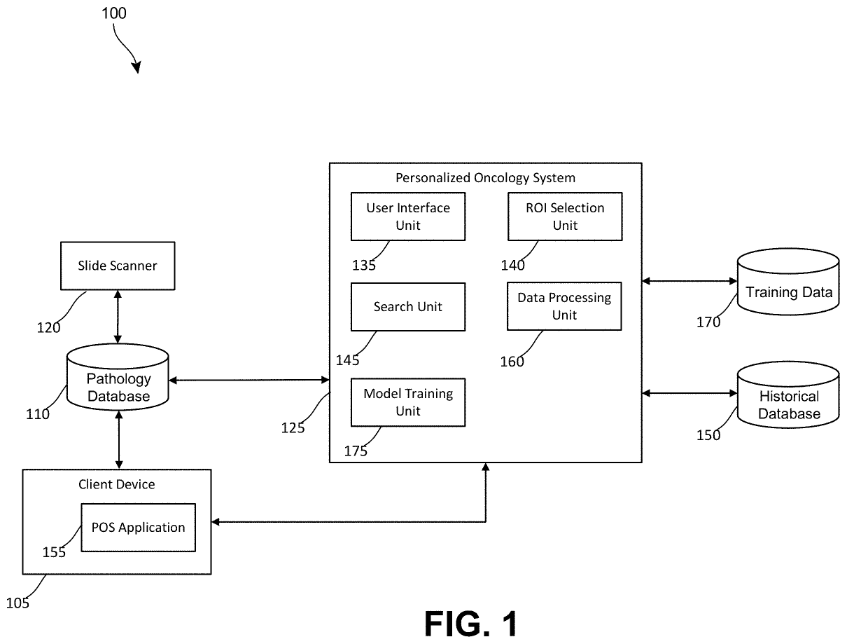

FIG. 1 is a block diagram showing an example computing environment 100 in which an example personalized oncology system 125 may be implemented. The computing environment 100 includes a slide scanner 120, a pathology database 110, a client device 105, and a historical database 150 in addition to the personalized oncology system 125. The personalized oncology system 125 may include a user interface unit 135, a regions-of-interest (ROI) selection unit 140, a search unit 145, and a data processing unit 160. The personalized oncology system 125 may also include a model training unit 175, and the computing environment 100 may include a training data store 170.

The slide scanner 120 may be used by the pathologist to digitize histopathology slides. The slide scanner 120 may be a whole-slide digital scanner that may scan each slide in its entirety. The slide scanner 120 may output a digital image of each slide that is scanned to the pathology database 110.

The client device 105 is a computing device that may be implemented as a portable electronic device, such as a mobile phone, a tablet computer, a laptop computer, a portable digital assistant device, and/or other such device. The client device 105 may also be implemented in a computing device having other form factors, such as a desktop computer, and/or other types of computing device. The client devices 105 may have different capabilities based on the hardware and/or software configuration of the respective client device. The example implementation illustrated in FIG. 1 includes a client device 105 that is separate from the personalized oncology system 125. In such an implementation, the client device 105 may access the personalized oncology system 125 over a network and/or over the Internet. The personalized oncology system 125 may include a personalized oncology system (POS) application 155 which may be configured to communicate with the personalized oncology system 125 to access the functionality provided by the personalized oncology system 125. In some implementations, the personalized oncology system 125 may be provided as a cloud-based service, and the POS application 155 may be a web-browser or a browser-enabled application that is configured to access the services of the personalized oncology system 125 as a web-based application. In other implementations the functionality of the personalized oncology system 125 may be implemented by the client device 105. In such implementations, the POS application 155 may implement the functionality of the personalized oncology system 125.

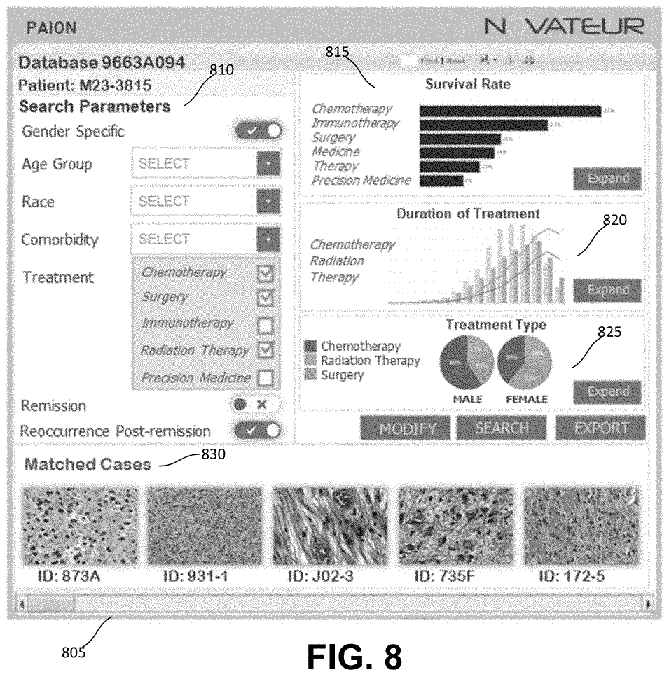

The user interface unit 135 may be configured to render the various user interfaces described herein, such as those shown in FIGS. 8, 9, and 10, which are described in greater detail in the examples that follow.

The ROI selection unit 140 allows a user to select one or more regions of interest in a histopathological image for a patient. The user may request the histopathological image may be accessed from the pathology database 110. The ROI selection unit 140 may provide tools that enable the user to select one or more ROI. The ROI selection unit 140 may also implement an automated process for selecting one or more ROI in the image. The automated ROI selection process may be implemented in addition to the manual ROI selection process and/or instead of the manual ROI selection process. Additional details of the ROI selection process are discussed in the examples which follow.

The search unit 145 may be configured to search the historical database 150 to find histopathological imagery stored therein that is similar to the ROI identified by the user. The search unit 145 may also allow the user to select other search parameters related to the patient, such as the patient's age, gender, ethnicity, comorbidities, treatments received, and/or other such criteria that may be used to identify data in the historical database 150 that may be used to generate a personalized therapeutic plan for the patient. As will be discussed in the various examples which follow, the search unit 145 may implement one or more machine learning models which may be trained to identify historical data that may be relevant based on the ROI information and other patient information provided in the search parameters. For example, the search unit may be configured to implement one or more deep convolutional neural networks (DCNNs).

The historical database 150 may store historical histopathological imagery that has been collected from numerous patients. The historical database 150 may be provided by a third party which is separate from the entity which implements the personalized oncology system 125. The historical database 150 may be provided as a service in some implementations, which may be accessed by the personalized oncology system 125 via a network and/or via the Internet. The histopathological imagery stored in the historical database 150 may be associated with clinical data, which may include information associated with the patient associated with the selected historical imagery, such as but not limited to diagnoses, disease progression, clinical outcomes, time-to-events information. The personalized oncology system 125 may search through and analyze the histopathological imagery and clinical data stored in the historical database 150 to provide a patient with a personalized therapeutic plan as will be discussed in greater detail in the examples which follow.

The model training unit 175 may be configured to use training data from the training data store 170 to train one or more models used by components of the personalized oncology system 125. The training data store 170 may be populated with data selected from one or more public histopathology data resources as will be discussed in the examples which follow.

The data processing unit 160 may be configured to implement various data augmentation techniques that may be used to improve the training of the models used by the search unit 145. The data processing unit 160 may be configured to handle both histology-specific variations in images as well as rotations in imagery. The data processing unit 160 may be configured to use one or more generative machine learning models to generate new training data that may be used to refine the training of the models used by the search unit 145. The data processing unit 160 may be configured to store new training data in the training data store 170. Additional details of the implementations of the models used by the data processing unit 160 will be discussed in detail in the examples that follow.

FIG. 8 is an example user interface 805 of the personalized oncology system 125 for searching the historical database 150. The user interface 805 may be implemented by the user interface unit 135 of the personalized oncology system 125. The user interface 805 may be configured to conduct the search given a slide image from the pathology database 110 and user-specified search parameters. The search parameters 810 allow the user to enter select a gender-specific indicator, an age group indicator, a race indicator, comorbidity information, treatment information, a remission indicator, and a recurrence after remission indicator. The gender-specific indicator limits the search to the same gender as the patient for whom the search is being conducted. The age group indicator represents an age range to which the results should be limited. The age range may be an age range into which the patient's age falls. The race information may be used to limit the search results to a particular race or races. The comorbidity information may be used to limit the search to include patients having one or more additional conditions co-occurring with the primary condition. The treatment information may allow the user to select one or more treatments that may be provided to the patient in order to search the histopathological database for information associated with those treatments. The remission indicator may be selected if the patient is currently in remission, and recurrence after remission indicator may be selected to indicate that the patient has experienced a recurrence of the cancer after remission.

The survival rate information 815 provides survival rate information for patients receiving each of a plurality of treatments. The survival rate information may be survival rates for patients that match the search parameters 810. The survival rate information 815 may include an “expand” button to cause the user interface 805 to display additional details regarding the survival rate information.

The duration of treatment information 820 displays information indicating how long each type of treatment was provided to the patient. In the example shown in FIG. 8, the duration of treatment information 820 may be displayed as a graph. The duration of treatment information 820 may include an “expand” button to cause the user interface 805 to display additional details regarding the duration of treatment information.

The treatment type information 825 may show a percentage of patients that received a particular treatment. The treatment type information 825 may be broken down by gender to provide an indication how many male patients and how many female patients received a particular treatment. The treatment type information 825 may include an “expand” button to cause the user interface 805 to display additional details regarding the treatments that were given to the patients.

The matched cases 830 include cases from the historical histopathological database. The matched cases 830 may include histopathological imagery that includes characteristics that the oncologist may compare with histopathological imagery of the patient. The matched cases 830 may show details of cases from the database that may help to guide the oncologist treating the patient by providing key insights and clinical outcomes based on the patient's own histological and other personal factors. The oncologist may use this information to identify an optimal therapeutic plan for the patient.

The histopathological imagery stored in the pathology database 110 and the historical database 150 play a critical role in the cancer diagnosis process. Pathologists evaluate histopathological imagery for a number of characteristics, that include nuclear atypia, mitotic activity, cellular density, and tissue architecture to identify cancer cells as well as the stage of the cancer. This information enables the patient's doctors to create optimal therapeutic schedules to effectively control the metastasis of tumor cells. Recent advent of whole-slide digital scanners for digitization of histopathology slides has further enabled the doctors to store, visualize, analyze, and share the digitized slide images using computational tools and to create large pathology imaging databases that continue to rapidly grow.

An example of such a pathology imaging database is maintained by Memorial Sloan Kettering Cancer Center (“MSKCC”). MSKCC may be used to implement the historical database 150. MSKCC creates approximately 40,000 digital slides per month. The average size of a digital slide is approximately 2 gigabytes of data. Thus, MSKCC may generate more than 1 petabyte of digital slide data over the course of the year at this single cancer center. Despite having access to this wealth of pathology imagery data, the utility of this data for cancel diagnosis and clinical decision-making is typically limited to that of the original patient due to a lack of automated methods that can effectively analyze the imagery data and provide actions insights into that data. Furthermore, the sheer volume of unstructured imagery data in the pathology imaging database and the complexity of the data present a significant challenge to doctors who wish to search the imagery database for content that may assist the doctors to provide an improved cancer diagnosis and therapeutic plan for treating their patients. Therefore, there is a long felt need to develop automated approaches for searching for and analyzing the data stored in the imagery database to provide improved cancer diagnosis and clinical decision-making for treating their patients.

Automated analysis of histology images has long been a topic of interest in medical image processing. Several approaches have been reported for grading, and identification of lymph node metastases in multiple cancer types. Early medical image analysis approaches heavily relied on hard-coded features. Some examples of these approaches are scale-invariant feature transform (SIFT), histogram of oriented gradients (HOG), and Gray-Level Co-Occurrence Matrix (GLCM). These early approaches are used to explicitly identify and describe structures of interest that are believed to be predictive. These features were then used to train classification models that predict patient outcomes. However, these traditional methods only achieved limited success (with reported accuracies around 80% to 90%) to be viable for diagnosis and treatment in clinical settings.

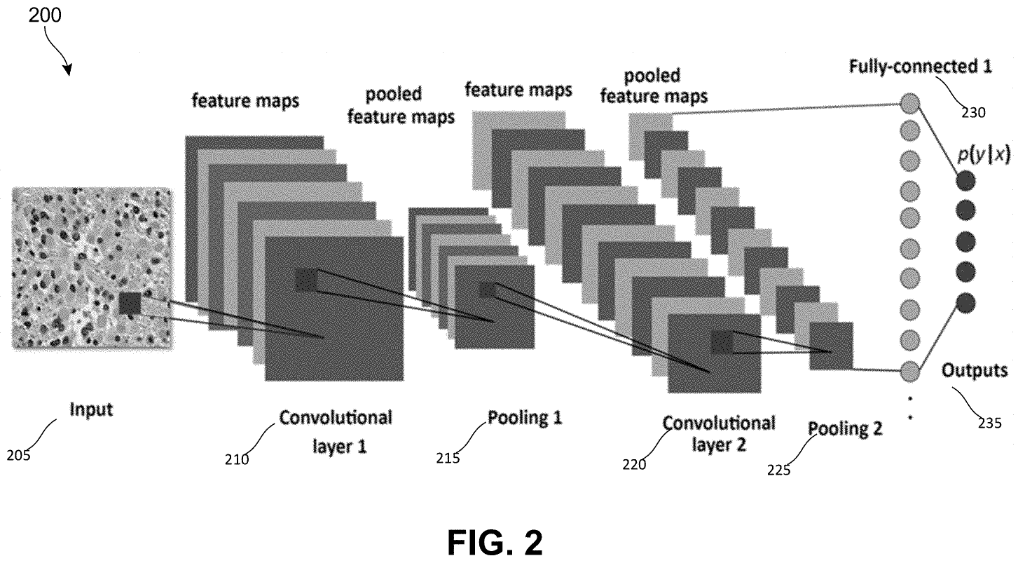

Recently supervised deep learning techniques and deep convolutional neural networks (CNNs) have shown remarkable success in visual image understanding, object detection, and classification, and have shattered performance benchmarks in many challenging applications. As opposed to traditional hand-designed features, the feature learning paradigm of CNNs adaptively learns to transform images into highly predictive features for a specific learning objective. The images and patient labels are presented to a network composed of interconnected layers of convolutional filters that highlight important patterns in the images, and the filters and other parameters of this network are mathematically adapted to minimize prediction error in a supervised fashion, as shown in FIG. 2.

FIG. 2 shows an example of the structure of an example CNN 200 which may be implemented by the search unit 145 of the personalized oncology system 125. The CNN 200 includes an input image 205, which is a histopathology image to be analyzed. The histopathology image may be obtained from the pathology database 110 for a patient for whom a personalized therapeutic plan is being developed. The input image 205 may be quite large and include a scan of an entire slide. However, as will be discussed in the examples which follow, “patches” of the input image 205 that correspond to one or more ROI identified by the user and/or automatically identified by the ROI selection unit 140 may be provided to the CNN 200 for analysis rather than the entire input image 205. This approach may significantly improve the results provided by the CNN 200 and may also significantly reduce the amount of training data required to train the CNN 200.

The first convolutional layer 210 applies filters and/or feature detectors to the input image 205 and outputs feature maps. The first pooling layer 215 receives the feature maps of the first convolutional layer 210 and operates on each feature map independently to progressively reduce the spatial size of the representation to reduce the number of parameters and computation in the CNN 200. The first pooling layer 215 outputs pooled feature maps which are then input to the second convolutional layer 220. The second convolutional layer 220 applies filters to the pooled feature maps to generate a set of feature maps. These feature maps are input into the second pooling layer 225. The second pooling layer 225 analyzes the feature maps and outputs pooled feature maps. These pooled feature maps are then input to the fully connected layer 230. The fully connected layer 230 is a layer of fully connected neurons, which have full connections to all activations in the previous layers. The convolutional and pooling layers break down the input image 205 into features and analyze these features. The fully connected layer 230 makes a final classification decision and outputs a label 235 that describes the input image 205. The example implementation shown in FIG. 2 includes only two convolutional layers and two pooling layers, but other implementations may include more convolutional layers and pooling layers.

Supervised feature learning, such as that provided by the CNN 200, avoids biased a priori definition of features and does not require the use of segmentation algorithms that are often confounded by artifacts and natural variations in image color and intensity. The ability of CNNs to learn predictive features rather than relying on hand-designed, hard-coded features has led to the use of supervised deep-learning based automated identification of disease from medical imagery. PathIA, Proscia, and Deep Lens are a few examples of companies that are applying machine learning and deep learning techniques to attempt to obtain more accurate diagnosis of disease.

While feature learning using deep convolutional neural networks (DCNNs) has become the dominant paradigm in general image analysis tasks, histopathology imagery poses unique technical problems that are difficult to overcome and still limit the applicability of supervised techniques in clinical settings as follows. These technical problems include: (1) insufficient data for training of the models, (2) the large size of histopathology images, (3) variations in how histopathology images are formed, (4) unstructured image regions-of-interest with ill-defined boundaries, (5) the non-Boolean nature of clinical diagnostic and management tasks, and (6) user's trust in black-box models for clinical applications. Each of these technical problems is explored in greater detail below before describing the technical solutions provided by the techniques provided herein.

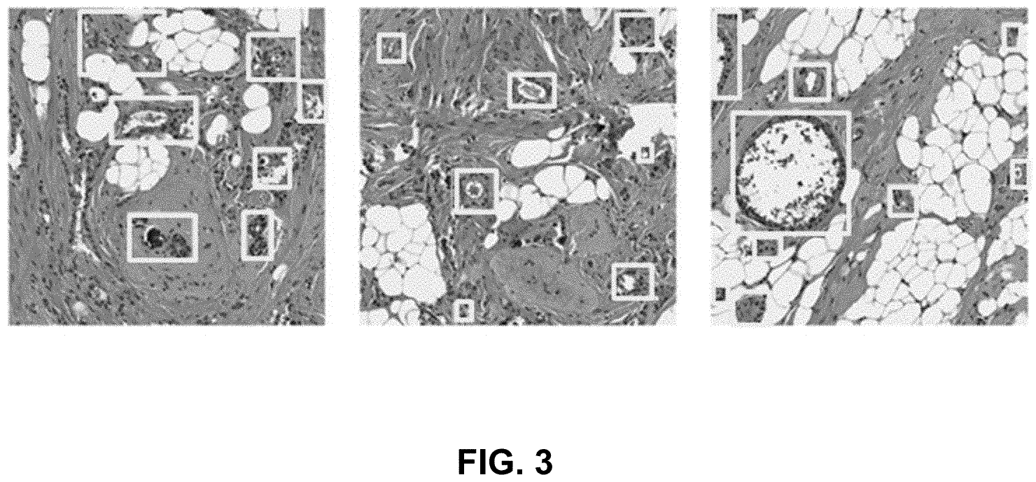

Insufficient data for training the models used by deep-learning is a significant problem that may limit the use of deep-learning for the analysis of histopathology imagery. The success of deep-learning approaches significantly relies on the availability of large amounts of training data to learn robust feature representations for object classification. Even pre-training the DCNN on large-scale datasets, such as ImageNet, and fine tuning the DCNN for the analysis of histopathology imagery requires tens of thousands of labeled examples of the objects of interest. However, the access to massive high-quality datasets in precision oncology is highly constrained. There is a relative lack of large truth or reference datasets containing carefully molecularly characterized tumors and their corresponding detailed clinical annotations. For example, the TUPAC16 (Tumor Proliferation Assessment Challenge) dataset has only 821 whole slide images from The Cancer Genome Atlas (“TCGA”). While TCGA has tens of thousands of whole slide images available in total, these images are only hematoxylin and eosin (H&E) stained slides and only contain clinical annotations, such as text reports that apply to the whole-slide image as opposed to specific regions of the image, as shown in FIG. 3. FIG. 3 provides an example in which bounding boxes have been placed around regions of interest in the images. While annotation of imagery for deep learning is a tedious task for any application, reliable annotation of medical imagery can only be obtained from highly trained doctors who are not only very expensive, but also are generally not interested or motivated to perform such a tedious task. Data augmentation techniques as well as synthetic data have been used to alleviate some of these challenges. However, these approaches are not a substitute for high-quality annotations on real imagery. Therefore, there is a real need to develop novel models and technologies for automated histopathology data analysis that do not rely on such availability of large amount of annotated imagery.

The large image size of presents another significant problem in histopathology imagery analysis. While many image classification and object detection models are capable of exploiting image-level labels, such as those found in the ImageNet dataset, to automatically identify regions of interest in the image, these models assume that the objects of interest, for which the labels are available, occupy a large portion of the image. In contrast, histopathology images are typically much larger than those found in other imaging specialties: a typical pathology slide digitized at high magnification can be as large as 100,000×100,000 pixels. Whereas a tumor in a pathology image may encompass only a few hundred pixels, a significantly small portion (about a millionth) of the total image area. While trained clinicians and researcher can visually search the image to locate the lesion, training a deep neural network model to identify these locations with such coarse annotation is extremely difficult as the network has no supervision as to which part of the image the label is referring. Furthermore, many pathology cases contain multiple images and do not generally have image-specific labels about the disease and its stage. These operational scenarios pose significant challenges to most existing deep learning approaches for image analysis and image-based prediction.

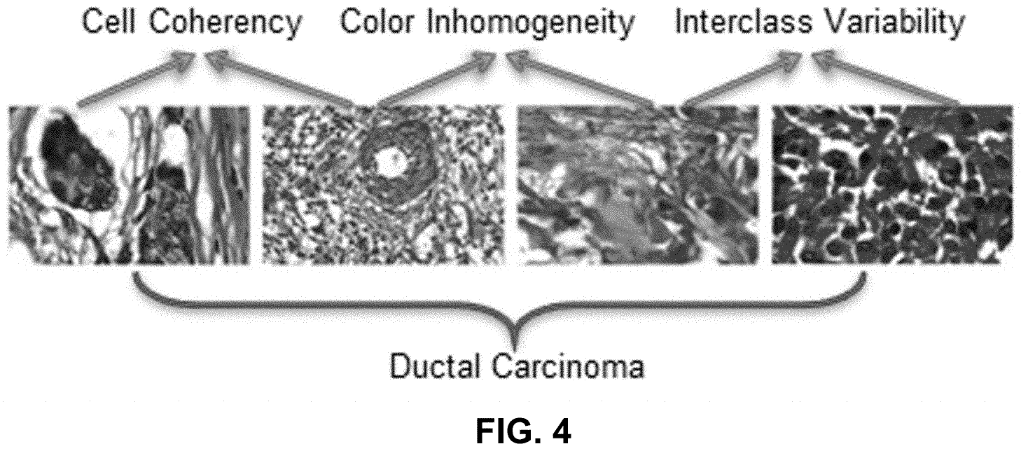

Variations in image formation processes present another significant problem in histopathology imagery analysis. Variations in staining color and intensity complicate quantitative tissue analysis. Example of such variations are shown in FIG. 4. FIG. 4 shows an example of cell coherency variations, color inhomogeneity variations, and interclass variability variations. Such variations are due to inter-patient variation, high coherency of cancerous cells, and inconsistencies in the preparation of histology slides (e.g. staining duration, stain concentration, tissue thickness). While one would expect the neural networks to learn these variations, once again, such learning would require a large number of training images from a variety of settings. Furthermore, the features learned by convolutional neural networks are generally not rotational invariant, and therefore require additional mechanisms to handle rotational variations in images.

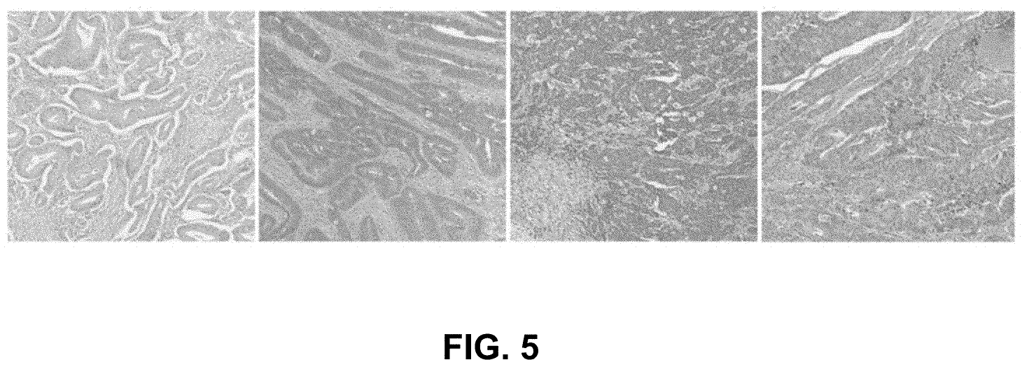

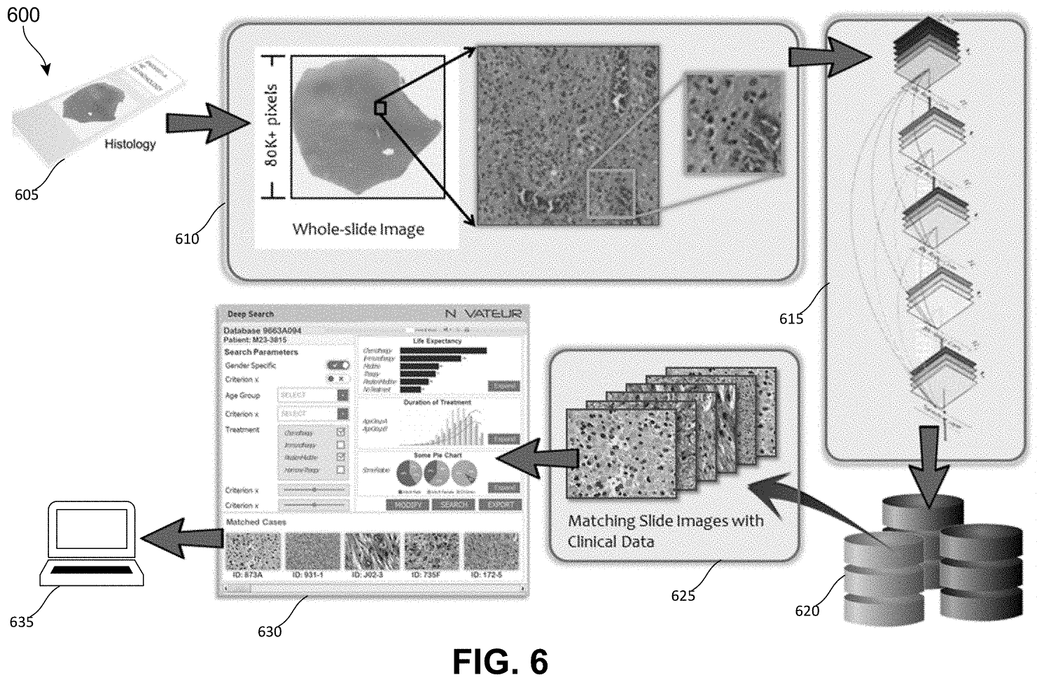

Unstructured image regions-of-interest with ill-defined boundaries present another significant problem in histopathology imagery analysis. One common approach in dealing with small amount of training data is transfer learning, where features learned from one domain (where large amount of data is already available) are adapted to the target domain using limited training samples from target domain. Typically, these methods use pre-trained networks from large image databases, such as ImageNet, COCO, etc. However, the images and object categories in these datasets are generally well structured with well-defined boundaries. In contrast, histopathology images and features may or may not be as well-structured. For example, many regions-of-interest (ROI) in histopathology images are characterized more by the texture-like features rather than presence of well-defined boundaries or structure in the imagery (e.g., FIG. 5). Noticeably, different classes have subtle differences and cancerous cells have high coherency. The features learned from ImageNet then do not transfer well to these cases. FIG. 6 shows example images of different grades of breast tumors, in which the images on the left show low-grade tumor and the images on the right show high-grade tumors. The examples shown in FIG. 5 do not have well-defined structure, in contrast with the example imagery from ImageNet which includes images that contain structured objects with well-defined boundaries.

The non-Boolean nature of clinical diagnostic and management tasks presents another significant problem in histopathology imagery analysis. As opposed to traditional classification problems, many important problems in the clinical management of cancer involve regression, for example, accurate prediction of overall survival and time-to-progression. Despite success in other applications, deep learning has not been widely applied to these problems. Some of the earlier work in this regard approached survival analysis as a binary classification problem, for example, by predicting binary survival outcomes (e.g., true/false) at a specific time interval (e.g., 5-year survival). However, this approach is limited as i) it is unable to use data from subjects with incomplete follow-ups and ii) does not allow probability of survival at arbitrary time values. Some of the more recent work tackled these limitations by adapting advanced deep neural networks to exploit time-to-event models such as Cox regression. However, due to the reasons outlined above, when predicting survival from histology, these approaches achieve only marginally better than random accuracy. The data challenges in time-to-event prediction are further intensified as (i) clinical follow-up is often difficult to obtain for large cohorts, and (ii) tissue biopsy often contains a range of histologic patterns (high intra-tumoral heterogeneity) that correspond to varying degrees of disease progression or aggressiveness. Furthermore, risk is often reflected in subtle changes in multiple histologic criteria that can require years of specialized training for human pathologists to recognize and interpret. Developing an algorithm that can learn the continuum of risks associated with histology can be more challenging than for other learning tasks, like cell or region classification.

The user's trust in black-box models for clinical applications presents another significant problem in histopathology imagery analysis. CNNs are black-box models composed of millions of parameters that are difficult to deconstruct. Therefore, the prediction mechanisms used by the CNNs are difficult to interpret. This is a major concern in a variety of applications that include autonomous vehicles, military targeting systems, and clinical predictions where an error by the system can be extremely costly. Moreover, the users (doctors) also find it difficult to trust black-box models and base their clinical decisions purely on machine predictions. This lack of transparency and interpretability is one of the major impediments in commercialization of deep learning-based solutions for clinical applications.

The personalized oncology system 125 addresses the serious technical, logistical, and operational challenges described above associated with developing supervised deep learning-based systems that analyze histopathological imagery for automating clinical diagnostics and management tasks. Furthermore, many of these technical problems, such as reliability and interpretability of deep learning models for clinical decision making and the reliance on availability of large amount of annotated imagery, are so fundamentally tied to the state-of-the-art in deep learning that it would require another major paradigm shift to enable fully-automated clinical management from histopathological imagery.

The personalized oncology system 125 disclosed herein provides technical solutions to the above-mentioned technical problems, by leveraging recent advances in deep learning, one-shot learning, and large-scale image-based retrieval for personalized clinical management of cancer patients. Instead of relying on a black-box system to provide the answers directly from histopathology images of a given patient, the success of an image-based clinical management system hinges upon creating an effective blend of (i) the power of automated systems to mine the vast amounts of available data sources, (ii) the ability of modern deep learning systems to learn, extract, and match image-features, and (iii) the perception and knowledge of a trained professional to identify the subtle patterns and shepherd the prediction and decision-making. The example implementation that follow describe the interaction between the pathologist and novel automated tools for knowledge discovery that enable finding informative features in imagery, pattern matching, data mining, and searching large databases of histopathology images and associated clinical data.

FIG. 6 shows a high-level workflow process 600 for providing a personalized therapeutic plan for a patient. The process 600 may be implemented by the personalized oncology system 125. As discussed in the preceding examples, the user interface unit 135 of the personalized oncology system 125 may provide a user interface that allows a user to conduct a search through the historical database 150 of histopathology images and associated clinical data to create a personalized therapeutic plan for a patient. The user is typically an oncologist, pathologist, or other doctor developing the therapeutic plan for the patient.

The process 600 may include an operation 605 in which a whole-slide image is accessed from the pathology database 110. As discussed in the preceding examples, the pathology database 110 may include whole-slide images of biopsy or surgical specimens taken from the patient which have been scanned using the slide scanner 120. The slide may then be scanned using a whole-slide digital scanner and stored in the pathology database. The user interface provided by the user interface unit 135 may provide a means for searching the pathology database 110 by a patient identifier, patient name, and/or other information associated with the patient that may be used to identify the slide image or images associated with a particular The user may select the whole-slide image from a pathology database 110 or other data store of patient information accessible to the personalized oncology system 105.

The process 600 may include an operation 610 in which the regions of interest (ROI) in the whole-slide image are selected. The user interface unit 135 of the personalized oncology system 125 may display the slide that was accessed in operation 605. The ROI selection unit 140 may provide tools on the user interface that enable the user to manually select one or more ROI. In the example shown in FIG. 6, the user may draw a square or rectangular region around an ROI. The ROI selection unit 140 may determine the coordinates of the selected ROI by mapping the square or rectangle drawn on the whole-slide image. The ROI selection unit 140 may also allow the user to draw other shapes around a ROI or draw a freehand shape around an ROI. The ROI selection unit 140 may also be configured to automatically detect one or more ROI. The system may include intelligent deep-learning based tools for segmentation and attention-based vision to assist the user in finding the ROI in a more efficient manner. The automated ROI search tools may be automatically invoked by the system when the whole-slide image is accessed or the user interface may provide a button or other user interface element that enables the user to invoke the automated ROI search tools. The automated ROI search tools may draw a border around each detected ROI similar to those which may be manually drawn around an ROI by the user. The ROI selection unit 140 may allow the user to deselect one or more of the ROI that were automatically selected by the automated ROI search tools. The ROI selection unit 140 may also provide means for manually adjusting the borders of the automated ROI by selecting a border of the ROI and dragging the border to cover a desired area. The ROI selection unit 140 may provide the user with an option to save the one or more ROI in the patient information associated with the slide in the pathology database to permit the user to later access the slide and view and/or manipulate the ROI associated with the slide. In some implementations, the user-selected ROI and the automatically-selected ROI may be highlighted using a different color, border pattern, and/or other indicator to permit the user to differentiate between the user-selected ROI and the automatically-selected ROI. The ROI selection unit 140 may also be configured to generate training data for the models used to automatically select ROI based on the user-selected ROI and/or update one or more operating parameters of the models based on the user-selected ROI. This approach may help improve the models used by the ROI selection unit 140 to automatically select ROI that are similar to those that were selected by the user but not automatically selected by the ROI selection unit 140.

The process 600 may include an operation 615 in which the regions of interest (ROI) of the whole-slide image are provided to a DCNN of the search unit 145 of the personalized oncology system 125 for analysis. The DCNN is configured to extract features from the selected ROIs and match these features with pre-indexed features from the historic imagery stored in the historical histopathological database in operation 620. The matching historical imagery and associated clinical data are obtained from the historical histopathological database in operation 625 and provided to the personalized oncology system 125 for presentation to the user. The associated clinical data may include information associated with the patient associated with the selected historical imagery, such as but not limited to diagnoses, disease progression, clinical outcomes, time-to-events information.

The process 600 may include an operation 630 of presenting the matched imagery from operation 620 on the user interface of the client device 105. The user interface may be similar to that shown in FIG. 8 and may permit the user to view the matched imagery and clinical data and/or filter the matched data based on various parameters, such as but not limited to age, gender, race, comorbidity information, treatment options, and/or other filter criteria. The user may select one or more of these filter criteria to filter the data obtain historical information from other patients at a similar stage of a disease. The user may also filter the historical data for information for other patients who were given a particular treatment to predict a likelihood of survival and time-to-event information for the patient for whom the therapeutic plan is being developed.

The search-based techniques provided by the personalized oncology system 125 solve several major technical problems associated with deep-learning based systems that attempt to perform clinical predictions using supervised training. One technical problem that the personalized oncology system 125 solves is that the techniques implemented by the personalized oncology system 125 do not require the large amounts of annotated training data that is required by traditional deep-learning approaches. The traditional deep-learning approaches rely heavily on the availability of large amounts of annotated training data, because such supervised methods must learn a complex function with potentially millions of learned parameters that analyze raw-pixel data of histopathology images to infer clinical outcomes. However, such large amounts of annotated data required to train the deep learning models is typically unavailable. The techniques implemented by the personalized oncology system 125 solve this technical problem by utilizing the expertise of the pathologist to identify the regions of interest (ROI) in a patient's histopathology imagery. The ROI, also referred to herein as a “patch” of a histopathology image, is a portion of the whole-slide image. The CNNs of the system may then (1) analyze and refine the ROI data and (2) match the refined ROI data with the histopathology imagery and associated clinical data stored in the historical database 150. Because the personalized oncology system 125 uses a smaller ROI or patch rather than a whole-slide image when matching the historical data of the historical database 150, much less pre-annotated training data is required to train the machine learning models used by the search unit 145 to find matching historical data in the historical database 150. As will be discussed in greater detail below, the personalized oncology system 125 may utilize a one-shot learning approach in which the model may learn a class of object from a single labelled example.

The personalized oncology system 125 also provides a technical solution for handling the large image sizes of histopathological imagery. Current deep learning-based approaches cannot effectively handle such large image sizes. The techniques provided herein provide a technical solution to this problem in several ways. First, the expertise of the pathologist may be leveraged in identifying ROI and/or intelligent deep-learning based tools for segmentation and attention-based vision may assist the user in finding the ROI in a more efficient manner. As a result, a large amount of irrelevant data from the whole-slide image may be discarded. Second, as will be discussed in greater detail in the examples that follow, the personalized oncology system 125 may exploit a novel approach for rare-object detection in large satellite imagery. This approach utilizes robust template matching in large imagery and indexing large imagery for efficient search.

The personalized oncology system 125 also provide a technical solution to the technical problem of lack of transparency of deep learning methods. The black-box nature of current deep learning methods is a major challenge in commercializing these approaches in high-risk settings. Pathologists and patients may find it difficult to trust a prediction system that does not provide any visibility about underlying decision-making process. The techniques disclosed herein provide a solution to this and other technical problems by providing a glass-box approach that emphasizes transparency into the underlying decision process. Rather than being a decision-maker, the personalized oncology system 125 acts as a facilitator that enables the pathologists to make informed decisions by providing them key data points relevant to their subject. Furthermore, the personalized oncology system 125 provides the pathologists with all the supporting evidence (in the form of historical imagery, matched regions-of-interest, and associated clinical data) so that they can make confident predictions and clinical decisions.

The techniques provided herein make histopathological imagery databases diagnostically useful. As opposed to supervised system that only utilize historic imagery that accompanies high-quality expert annotations, the search-based approach of the personalized oncology system 125 enables exploitation of large histopathology image databases and associated clinical data for clinical diagnosis and decision-making. A technical benefit of the personalized oncology system 125 over traditional deep learning-based systems is that the personalized oncology system 125 enables personalized medicine for cancer treatment. Healthcare has traditionally focused on working out generalized solutions that can treat the largest number of patients with similar symptoms. For example, all cancer patients who are diagnosed with a similar form of cancer, stage, and grade are treated using the same approach, which may include chemotherapy, surgery, radiation therapy, immunotherapy, or hormonal therapy. This is partly because there currently are limited options for doctors that enable them to identify a priori whether a treatment would work for a patient or not. Therefore, doctors typically follow a standardized and most common approach for cancer treatment. The personalized oncology system 125 enables oncologists to shift from this generalized treatment approach and move towards personalization and precision. By effectively exploiting historic histopathology imagery and finding the health records that best match the patient's histology as well as other physical characteristics (age, gender, race, co-morbidity, etc.), the personalized oncology system 125 provides oncologists actionable insights that include survival rates, remission rates, and reoccurrence rates, of similar patients based on different treatment protocols. The oncologists can use these insights to (i) avoid unnecessary treatment that is less likely to work for the given patient, (ii) avoid side effects, trauma, and risks or surgery, and (iii) determine optimal therapeutic schedules that are best suited for the cancer patient.

The personalized oncology system 125 provides the technical benefits discussed above and address the limitations of the state-of-the-art in deep learning and content-based image retrieval (CBIR). A discussion of the technical limitations of current CBIR techniques and the improvements provided by the personalized oncology system 125 that address the shortcomings of these CBIR techniques.

Many techniques have been proposed for CBIR of medical imagery in general and histopathological imagery in particular in recent years. These techniques range from simple cross-correlation to hand-designed features and similarity metrics to deep networks of varying complexities. CBIR has been long dominated by hand-crafted local invariant feature-based methods, led by SIFT (and followed by similar descriptors such as speeded up robust features (SURF), Binary Robust Independent Elementary Features (BRIEF), Oriented FAST and Rotated BRIEF (ORB), and other application-specific morphological features). These methods provide decent matching performance when the ROI in the query image and the imagery in database are quite similar in appearance. However, these methods have several drawbacks when applied to general settings in intra-class variations (e.g., those shown in FIG. 4). While local features are somewhat invariant, they do not generalize well across large variations in data. Moreover, they are limited to mostly structured objects with well-defined edge surfaces and are not very suitable for matching a wide variety of histological patterns (e.g., those shown in FIG. 5). To handle this issue, texture-based representations (such as GLCMs, Gabor filters, and Steerable filters) have also been proposed. However, these representations do not generalize well to cases where morphology does provide key characteristics. Recently some deep learning-based approaches have also been applied to the problem of CBIR. However, these approaches also suffer from several limitations that must be resolved to enable a practical CBIR based personalized oncology system. These limitations include: the use of deep networks pre-trained on natural image datasets, the use of arbitrary distance metrics, the training methodology used by deep-embedding networks to learn similarity is not suitable for patch matching, the similarity learned by deep-embedding networks is not suitable for histology imagery, and inadequate handling of rotations and magnifications, and inadequate measure of success. The personalized oncology system 125 provides a technical solution that addresses these limitations.

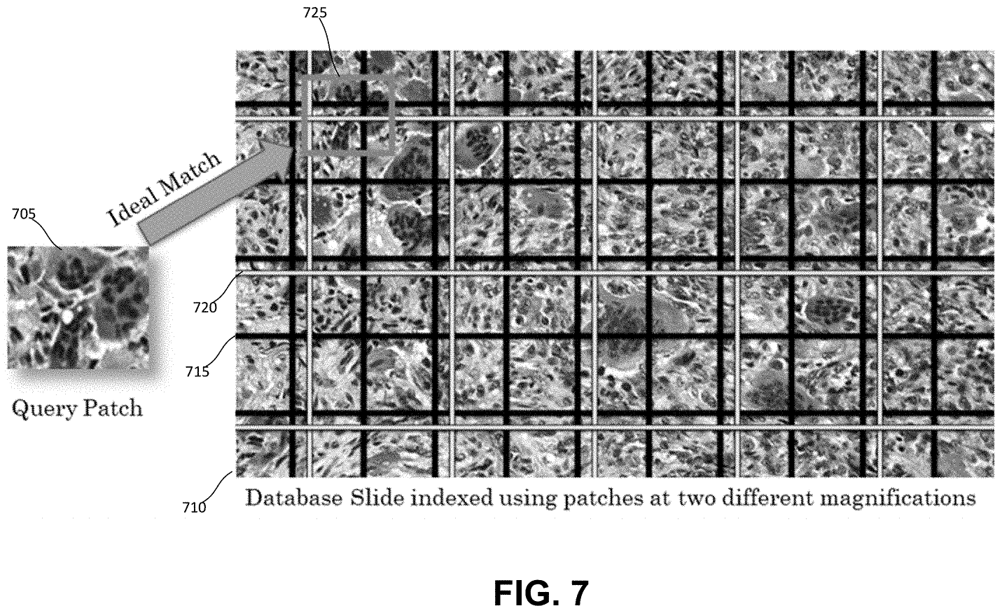

Existing deep learning based CBIR approaches for histopathology imagery use deep convolutional networks that are pre-trained on natural image datasets, such as ImageNet. There are two significant drawbacks with this approach. First, as discussed earlier, the features learned from natural images of well-structured objects do not correspond well to the features in histopathological imagery (see e.g., FIGS. 6 and 7). Second, the features are typically learned for the specific task of image classification on the ImageNet dataset, and thus, are not necessarily suitable for the task of retrieval of histopathological imagery.

Existing CBIR approaches also use arbitrary distance metrics. Except for Similar Medical Images Like Yours (SMILY), almost all the existing approaches simply use deep convolutional neural networks as a feature extractor and then apply traditional distance measures, such as L2 distance between computed features, to find similarity. However, this approach is not reliable as the arbitrary distance of high dimensional feature vectors do not necessarily correspond to the cognitive similarity of imagery.

The training methodology used by deep-embedding networks to learn similarity is not suitable for patch matching (or ROI matching) as described herein. Among the existing deep learning-based system, SMILY uses deep embedding networks for similarity matching. Deep embedding networks or metric learning methods attempt to learn a representation space where distance is in correspondence with a notion of similarity. In other words, these deep embedding networks or metric learning methods use large amount of labeled training data in an attempt to learn representations and similarity metrics that allow direct matching of new input to the labeled examples in the similar vein as template matching in a generalized and invariant feature space. The DCNN-based metric learning approaches, such as MatchNet and the deep ranking network used by SMILY, generally rely on a two-branch structure inspired by Siamese neural networks (also referred to as “twin neural networks”). Siamese neural networks are given pairs of matching and nonmatching patches and learn to decide whether the patches in the network match each other. These methods offer several benefits. For example, they enable zero-shot learning, learn invariant features, and gracefully scale to instances with millions of classes. The main limitation of these methods is due to the way they assess similarity between the two images. All metric learning approaches must define a relationship between similarity and distance, which prescribes neighborhood structure. In existing approaches, similarity is canonically defined a-priori by integrating available supervised knowledge, for example, by enforcing semantic similarity based on class labels. However, this collapses intra-class variation and does not embrace shared structure between different classes.

Another limitation of existing techniques is that similarly learned by deep-embedded networks is not suitable for histology imagery. Another limitation of the embedding network using in SMILY is that the network is once again trained on natural images (cats, dogs, etc.) and therefore suffers from the above-mentioned challenges. Furthermore, due to this training methodology, the learned similarity is not tied to the problem at hand. In other words, the learned similarity of natural object classes does not necessarily capture the peculiarities of matching regions-of-interest in histology. Therefore, the network is unable to handle variations that are typical to histopathological data, e.g., variations due to differences in staining, etc.

Another limitation of existing solutions is inadequate handling of rotations and magnifications. As mentioned earlier, convolutional neural networks are not rotation invariant. To tackle this challenge, SMILY simply computes similarity on four 90-degree rotations and their mirror images. This approach not only increases the database size (by 8×), it also significantly increases the potential for false matches. Moreover, SMILY handles different magnifications (of input patches) by indexing non-overlapping patches of various magnifications (×40, ×20, ×10, etc.). Only ×10 magnification patches were used for most of the evaluation. Once again, this strategy is flawed because of (i) arbitrary quantization of patches, (ii) missing data at different magnifications (due to non-overlapping patches). They can use overlapping patches for real use-cases, however, like rotation-handling, doing so will increase the complexity of database as well as the potential for false matches. These technical problems are inherent to the underlying random patch-based indexing approach used by SMILY, as there is no good way of chopping a large slide into small patches without losing information, such as, magnifications and neighboring features. However, it is impossible to know in advance which features and/or magnifications will be needed for a given search.

FIG. 7 illustrates an example of inadequate handling of magnifications. FIG. 7 shows a query patch 705 being matched to a database slide 705 indexed using patches at two different magnification. The grid 715 represents a first magnification and the grid 720 represents a second magnification. The query patch 705 may not be properly matched with the ideal match 725 due to loss of features at the grid boundaries.

Another limitation of existing solutions is that they provide an inadequate measure of success. A major difference between the personalized oncology system 125 and other CBIR techniques is how the success of the system is measured. Most of the existing CBIR systems measure success based on whether they find a good match for a given image. For example, SMILY uses the top-five score, which evaluates the ability of their system to correctly present at least one correct result in the top-five search results. While such a metric is suitable for traditional CBIR techniques and Internet searches where the users are satisfied as-long-as the search-results contain at least one item of their interest, this is not true for the use-case of clinical decision making outlined as discussed in the preceding examples. In clinical applications, finding one matching slide is not very useful, even if it is a perfect match. This is because (i) similar histology does not necessarily imply that other clinical data is consistent, (ii) it only provides one data point and is not very informative for the purpose of prediction of survival and time-to-outcome, or selection of treatments, which requires system to not only retrieve a large number of high quality matches but also score them appropriately.

The personalized oncology system 125 addresses the technical problems associated with the current CBIR systems discussed above. The technical solutions provided by the personalized oncology system 125 include: (i) a novel deep embedding network architecture and training methodology for learning histology-specific features and similarity measures from unlabeled imagery, (ii) data augmentation techniques for histology imagery, (iii) techniques for efficient indexing and retrieval of whole-slide imagery, and (iv) intuitive user-interfaces for pathologists.

The novel deep embedding network architecture and training methodology for learning histology-specific features and similarity measures from unlabeled imagery is one technical solution provided by the personalized oncology system 125 that provides a solution to some of the technical problems associated with current CBIR systems. The search unit 145 of the personalized oncology system 125 includes a novel deep embedding network architecture along with a training approach that enables learning of domain-specific informative features and similarity measure from large amount of available histopathology imagery without the need for supervisory signals from manual annotations. The proposed approach treats the problem of computing patch matching similarity as a patch-localization problem and attempts to learn filters from unlabeled histology imagery that maximize the correlation responses of the deep features at the matched locations in the image.

The data processing unit 160 of the personalized oncology system 125 may be configured to provide data augmentation techniques for histology imagery. The personalized oncology system 125 may be configured to handle both histology-specific variations in images as well as rotations in imagery. The personalized oncology system 125 improves the training of deep embedding networks through novel data augmentation techniques for histology imagery. Typically, data augmentation techniques use pre-defined and hand-coded sets of geometric and image transformations to artificially generate new examples from a small number of training examples. In contrast, the data processing unit 160 of the personalized oncology system 125 may be configured to use deep networks, such as generative adversarial networks (GANs) and auto-encoder networks to learn generative models directly from histology imagery that encode domain-specific variations in histology data and use these networks to hallucinate new examples. The use of deep networks for data augmentation has several advantages. Since the deep networks learn image transformations directly from large number of histology images, they can learn models of a much larger invariance space and capture more complex and subtle variations in image patch representations.

The search unit 145 of the personalized oncology system 125 may also provide for efficient indexing and retrieval of whole-slide imagery. The personalized oncology system 125 uses indexing of whole-slide imagery (as opposed to patch-based indexing used in current approaches). This indexing can be done by (i) computing pixel-level deep features with granularity as defined by the stride of the network for the whole slide image, (ii) creating a dictionary of deep features by clustering the features of a large number of slides, and (iii) indexing the locations in slide images using the learned dictionary. To enable searching the image database at arbitrary magnification levels, features may be computed at different layers of the deep networks (corresponding to different retinal fields or magnifications). These multi-scale features can be indexed separately and will be retrieved based on the magnification of the query patch. The search unit 145 of the personalized oncology system 125 may also use additional techniques and software systems for efficient retrieval of relevant slides and associated clinical data based on the features computed from the query patch.

Existing public histopathology data resources may be identified and leveraged for the development of the various models used by the personalized oncology system 125. Several histopathology image analysis and CBIR systems have published their results using The Cancer Genome Atlas (TCGA) database. There are many slide images available in TCGA that can be used for training the models described herein. The slides available in the TCGA data portal are frozen specimens, which are not suitable for computational analysis. Instead, the Formalin-Fixed Paraffin-Embedded (FFPE) slides for corresponding patients that can also be downloaded from the TCGA repository. Other high-quality histopathology databases that may also be utilized to obtain data that may be used to train the models include but are not limited to: i) Digital Pathology Association (DPA)'s Whole Slide Imaging Repository that includes Johns Hopkins Surgical Pathology “Unknowns” case conference series spanning over 2000 whole slide images with meta-data on the diagnosis and clinical context; and ii) Juan Rosai's Collection that comprises digital images of original slide material of nearly 20,000 cases. The data identified from these resources may be used to train deep embedding networks as well as deep generative networks to learn histology-specific features as well as similarity metrics for patch matching which will be described in greater detail in the examples which follow. The whole slide imagery along with associated clinical metadata may be indexed in a database using the learned histology feature dictionaries as discussed in the examples which follow. The training data may be stored in the training data store 170.

The following examples provide additional details of the visual search and image retrieval infrastructure provided by the personalized oncology system 125. The personalized oncology system 125 may be implemented using an infrastructure that includes several containerized web services that interact to perform similarity search over a large corpus of imagery data, such as that stored in the historical database 150. The historical database 150 may be implemented as a SQL database, and the search unit 145 may implement a representational state transfer (REST) backend application programming interface (API). The user interface unit 135 may provide a web-based frontend for accessing the image processing service provided by the personalized oncology system 125. The image processing service may be implemented by the search unit 145 of the personalized oncology system 125. The historical database 150 may be configured to store the spatial features from the images that that may be searched/localized over as well as to keep track of the provenance of each feature, metadata associated with each image, and cluster indices for efficient lookup. The backend API may be configured to operate as a broker between the user and the internal data maintained by the personalized oncology system 125. The image processing service is the backbone of the search infrastructure and may be implemented using various machine learning techniques described herein. The image processing service may be configured to receive an image, such as the image 605, and to perform a forward pass through the deep learning model described in the examples which follow to extract features from the image. The large-scale search/localization may then proceed in two steps: (1) data ingestion and indexing, and (2) query. These steps will be described in greater detail in the examples which follow.

The personalized oncology system 125 provides a highly modularized design for addressing the challenges presented by the visual search and localization problem. As will be discussed in the examples which follow, the improvements to the feature extractor model may be easily integrated into the personalized oncology system 125 by swapping out the implementation of the image processing search with a different implementation. Furthermore, any improvements in the clustering algorithm or organization of the features extracted from the images may be used to reindex the existing historical database 150.

If the personalized oncology system 125 is warmed up with large amounts of existing data, then new data can easily be incorporated through the backend API and be immediately available for search. The frontend web service displays this functionality to the user in a web interface that can be iterated on and improved through feedback and testing as will be discussed in the examples which follow.

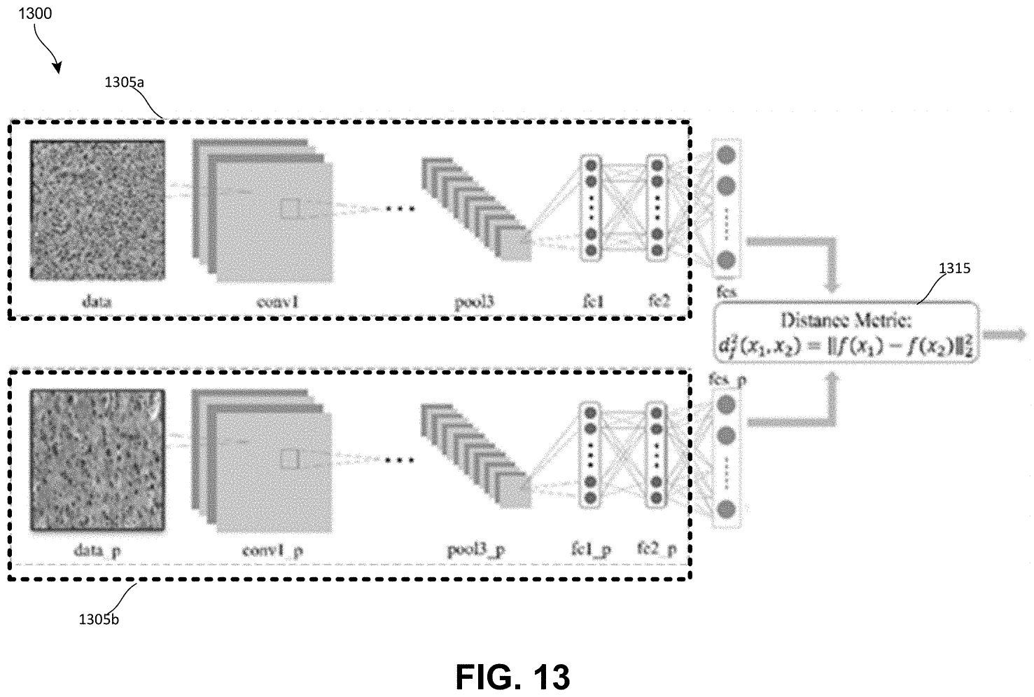

The personalized oncology system 125 may implement a novel deep embedding network architecture that is capable of learning domain-specific informative features and similarity measures from unlabeled data. Deep embedding networks (metric learning) attempt to learn a feature space (from large training datasets) along with a distance metric in the learned space that enable inference on whether two images are similar. That is, given a set of image pairs {(Iₐ,J_{b})}_{I}_{ₐ}_{,J}_{_{b}}_{∈I} the goal is to identify a feature embedding ϕ and a distance metric d, such that d(ϕ(Iₐ), ϕ(J_{b})) is small for matching pairs and large for non-matching pairs. FIG. 13 illustrates an example this concept in which an example Siamese network 1300 is shown. Siamese networks use DCNNs, such as the DCNNs 1305a and 1305b, to learn an embedding ϕ by minimizing a loss function, for example, contrastive loss: [symbol] (Iₐ,I_{b},l)=l*d(ϕ(Iₐ),f(I_{b}))+(1−l)*max(0,m−d(ϕ(Iₐ),f(I_{b})), where m is a margin parameter that omits the penalization if the distance of non-matched pair is big enough and l is a training parameter that identifies the matching and non-matching images. There are many other variants based on how the loss is defined and how the embeddings are learned using different configurations of the DCNNs. The assumption is that, once the embedding is learned, the distance property 1315 will remain valid on unseen pairs of images. Most of the existing work on image similarity learning enforces category-level image similarity (or semantic similarity) for training, i.e., two images are considered similar (l=1) as long as they belong to the same category. However, this approach collapses the intra-class variations, even though many semantic labels are known to have considerable visual variability within the class. Moreover, this approach requires object-level annotations in the training data.

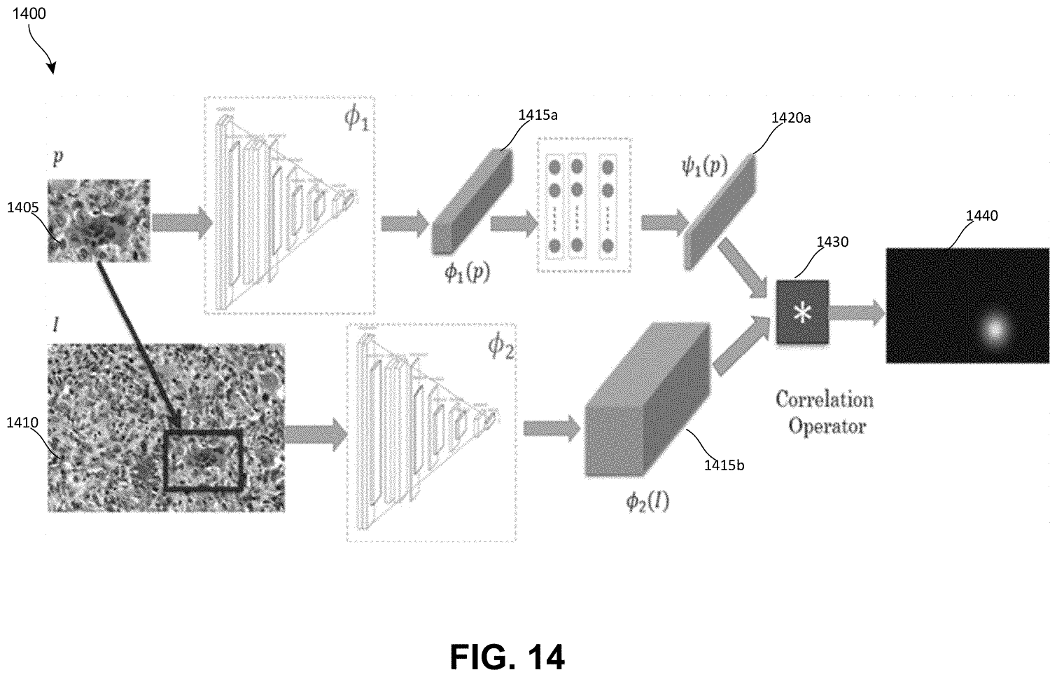

To address the deficiencies of the current approach, the techniques provided herein treat the problem of similarity learning in the context of patch-localization in an image. In other words, the Siamese network 1400 may be trained to locate an exemplar image within a larger search image. The high-level architecture of the proposed network is shown in FIG. 14. Specifically, given an image patch 1405, p, and a large search image 1410, I, we want the deep network to output a correlation map (or a likelihood map) such that the correlation map has high score at the locations in the search image with high similarity to the given patch. To achieve this, we use convolutional embedding functions (like Siamese networks shown in FIG. 13) that transforms the image data into a feature space where similarity can be easily computed. Typically, the two branches of the Siamese networks (convolutional neural networks) share the weights, i.e., they use the same embedding function for both the query and the search image and compute the distance directly on the spatial features computed by the convolutional neural networks. However, having spatial information in the query makes it much more difficult to achieve rotation and scale invariance in similarity learning. Therefore, in our case, we severe the weight-sharing between the branches and use an additional fully connected network that collapses the query image into a single non-spatial feature as shown in FIG. 14. The resulting feature maps ψ₁(p) 1420a and ϕ₂(I) 1415b, are combined using a cross correlation layer 1430, ψ₁(p)*ϕ₂(I). The output of the network is a score map 1440, f(p, I)→ [symbol] ² defined on a finite 2D grid as shown in FIG. 14 where the value of the map f(x, y) corresponds to the likelihood that the patch p matches the image I at locations corresponding to (x, y). The size of f is smaller than the size of I and is based on the size of the embedding network and network parameters.

The Siamese network 1400 may be trained using unlabeled histopathology imagery as follows. For positive patch-image pairs, patches may be randomly selected from imagery, and the network 1400 may be trained to match the patch from the image from which the patch is taken from with high confidence. To make the network resilient to common variations in histology image, we use data augmentation techniques, which are described in greater detail in the examples which follow, to transform the given patch and find the transformed patch in the original image. For negative patch-image pairs, the network 1400 can be shown a patch and an image that does not contain the patch. Without annotated data, this can be done by intelligently choosing images and patch pairs in a way that minimizes random chance of finding a matching pair, for example, by choosing pairs from different domains and scenarios, or by using low-level feature analysis.

Using the positive and negative training pairs, the network 1400 may use CenterNet loss to learn both the embedding functions as well as the correlation in an end-to-end fashion. This CenterNet loss is a penalty-reduced pixel-wise logistic regression with focal loss.

$${L}_{k} = \frac{- 1}{N} {\sum }_{xyc} \{ \begin{matrix}{( 1 - {\hat{Y}}_{xyc} )}^{α} log ( {\hat{Y}}_{xyc} ) & if {Y}_{xyc} = 1 \\ \begin{matrix}{( 1 - {Y}_{xyc} )}^{β} {( {\hat{Y}}_{xyc} )}^{α} \\ log ( 1 - {\hat{Y}}_{xyc} )\end{matrix} & otherwise\end{matrix}$$

where Y_{xyc} is a heatmap created by using a Gaussian kernel over the locations of the input patch in the search image, Ŷ_{xyc} is the output map from the network, and α and β are the hyper-parameters of the focal loss. The use of CenterNet loss drives the network to have a strong response only on pixels close to the center of an object of interest. This approach further reduces the difficulties in finding rotational/scale invariant representations, as the techniques disclosed herein are concerned only with getting a “hit” on the center of the query patch.

The personalized oncology system 125 disclosed herein may implement deep generative models for data augmentation of histology imagery. Human observers are capable of learning from one example or even a verbal description of the example. One explanation of this ability is that humans can use the provided example or verbal description to easily visualize or imagine what the objects would look like from different viewing points, illumination conditions, and other pose variations. To visualize new objects from different perspectives, humans use prior knowledge about the observations of other known objects and can seamlessly map this knowledge to new concepts. For instance, humans can use the knowledge of how vehicles look when viewed from different perspective to visualize or hallucinate novel observations of a previously unseen vehicle. Similarly, a child does not need to see examples of all possible poses and viewing angles when he/she learns about a new animal, rather they can leverage a priori knowledge (latent space) about known animals to infer how the new animal would look like at different poses and viewing angles. This ability to hallucinate novel instances of concepts can be used to improve the performance of computer vision systems (by augmenting the training data with hallucinated examples).

Data augmentation techniques are commonly used to improve the training of deep neural networks. Traditionally, this involves generation of new examples from existing data by applying various transformations to the original dataset. Examples of these transformations include random translations, rotations, flips, polynomial distortions, and color distortions. However, in real-world histopathology data, a number of parameters, such as coherency of cancerous cells, staining type and duration, and tissue thickness result in a large and complex space of image variations that is almost impossible to model using hand-designed rules and transformations. However, like human vision, given enough observations from the domain-specific imagery, common variations in image observations can be learned and applied to new observations.

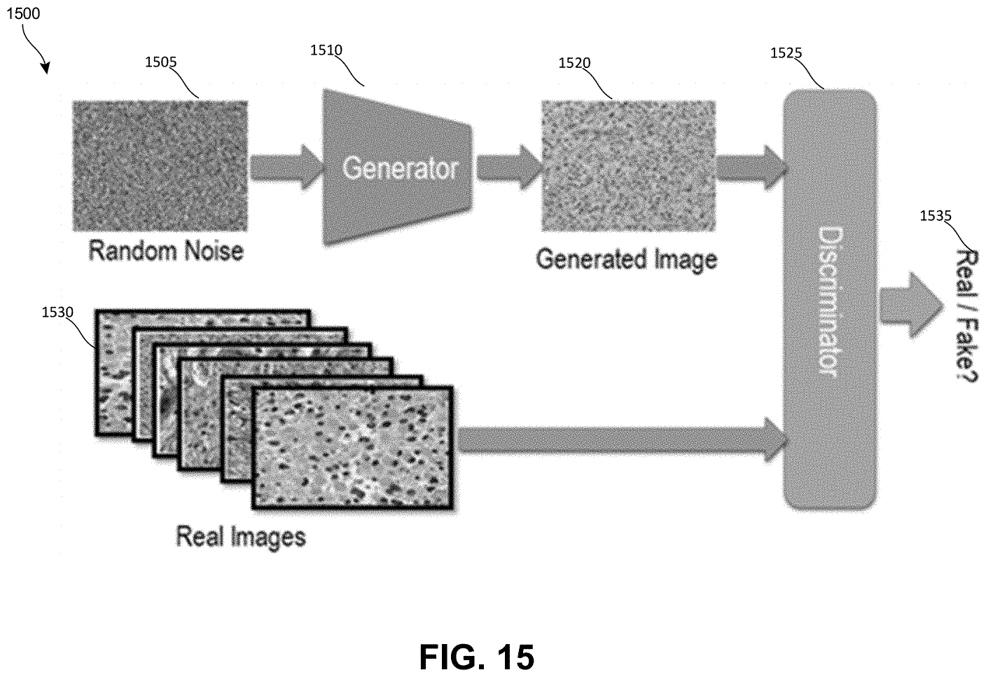

Recently, Generative Adversarial Networks (GANs) have gained popularity to learn the latent space of image observations and to generate novel examples from the learned space. FIG. 15 is a diagram of an example GAN 1500. GANs, such as the GAN 1500, are generative deep models that pit two networks against one another: a generative model 1510 G that captures the data distribution and a discriminative model 1525 D that distinguishes between samples drawn from model 1520 G and images drawn from the training data by predicting a binary label 1535. The generative model 1510 can be thought of as analogous to a team of counterfeiters, trying to produce fake currency and use it without detection, while the discriminative model 1525 is analogous to the law enforcement, trying to detect the counterfeit currency. Competition in this game drives both teams to improve their methods until the counterfeits are indistinguishable from the genuine articles. The networks 1510 and 1525 are trained jointly using backprop on the label prediction loss in a mini-max fashion: simultaneously update model 1510 G to minimize the loss while also updating model 1525 D to maximize the loss (fooling the discriminator). Formally, generative adversarial learning can be seen as an approach to generate examples from density that matches the density of a training dataset [symbol] ={x₁,x₂, . . . , x_{N}}. They learn this by minimizing a distribution discrepancy measure between the generated data and the true data. The generative model 1510 learnt by a GAN takes the form: z= [symbol] (0,I),v=f(z), where f is implemented via a neural network, v are the vectors being generated (that in distribution, should match the data [symbol] ), and z are the latent Gaussian variables that provide the variation in what is generated.

In the example shown in FIG. 15, the generator model 1510 may be provided an input 1505 that comprises white noise. The generator mode 1510 may generate a generated image 1520 based on the input 1505. The discriminator model 1525 may compare the generated image 1520 to real images 1530 to output a prediction 1535 whether the generated image 1520 is real or fake.

It has been shown that GANs are capable of learning latent spaces directly from imagery and generate photorealistic images. Since a large amount of (unlabeled) histopathology imagery is already available, there is not a need to generate new histopathology images. Instead, the personalized oncology system 125 may leverage GANs to learn natural variations in histopathology imagery to modify existing patches in realistic fashion to enable robust similarity learning. This can be done using a couple of different approaches that may be implemented by the personalized oncology system 125.

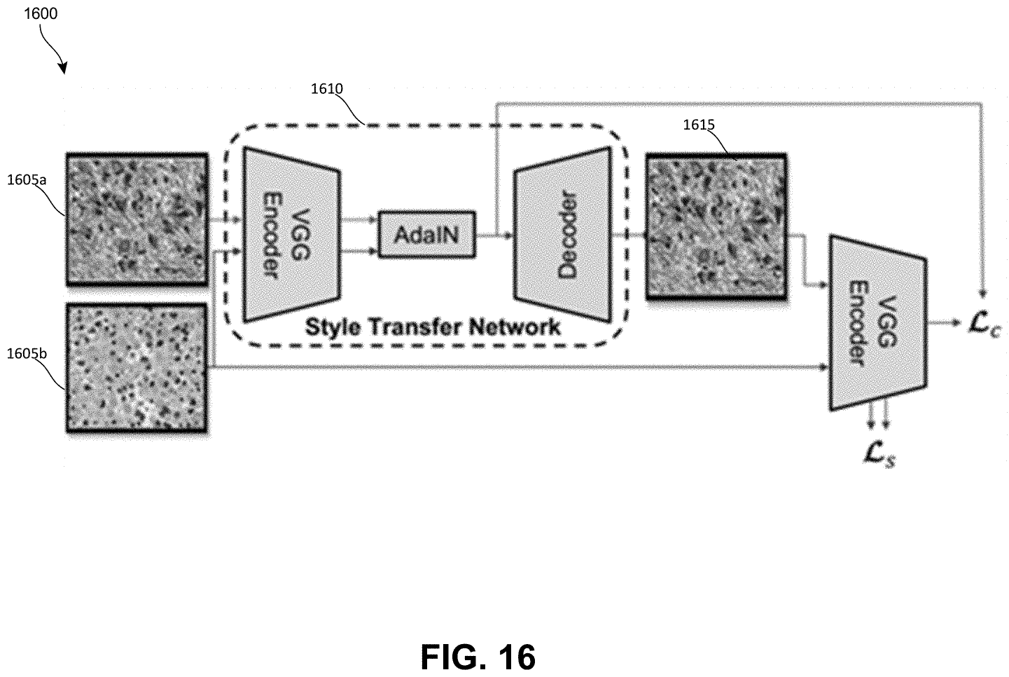

A first approach is to train style-transfer GANs using histology images. Instead of generating a brand new image from a random image (as in the example shown in FIG. 15), style-transfer GAN takes two images as input and transfers some of the high-level properties (style) while retaining low-level properties of the original image. Therefore, Style-Transfer GAN can be used to modulate the training patches to simulate variations in staining and other image-capture properties. FIG. 16 is a diagram showing an example of a style-transfer GAN 1600. The style-transfer GAN 1600 may be used to augment data based on image variations due to staining and/or other image capturing variables. The GAN 1600 takes two images 1605a and 1605b as input to the style transfer network 1510. The style transfer network 1610 outputs image 1615, which includes some of the high-level properties (style) of image 1605b while retaining low-level properties of the original image 1605a.

A second approach is to use a recently proposed style-based generator architectures that combine the properties of traditional GANs and style-transfer GANs to learn latent spaces that allow control of image synthesis process at varying degrees of freedom. This architecture uses multiple style-transfer GANs at different scales, which leads to automatic, unsupervised separation of high-level attributes (e.g., staining) from stochastic variation (e.g., small variations in morphology) in the generated images, and enables intuitive scale-specific mixing and interpolation operations. The style-based generators discussed in these examples may be used by the personalized oncology system 125 to generate novel imagery for training and obtaining augmentations by varying the imagery by changing the control parameters of the latent space.

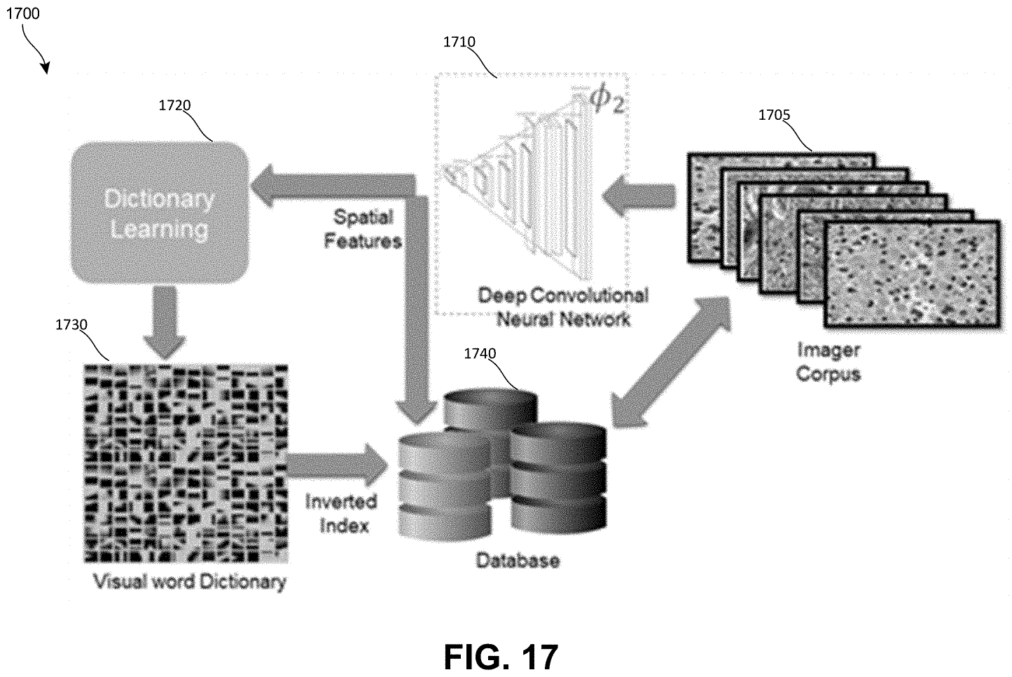

The personalized oncology system 125 may be configured to provide for efficient indexing and retrieval of whole-slide imagery from the historical database 150. The search infrastructure described in the preceding examples may be leveraged to facilitate the efficient indexing processes. The process 1700 may be used to create the historical database 150 and/or to add new imagery data to the historical database 150. The process 1700 may be implemented by the data processing unit 160 of the personalized oncology system 125. FIG. 17 is a diagram showing an example process 1700 for feature computation, dictionary learning, and indexing of the image corpus of the historical database 150. The process 1700 may be computationally intensive but needs only be performed once on the image corpus. The process 1700 includes two indexing steps or stages. In a first indexing step, a corpus of imagery 1705 is identified over which the user would like to conduct searches and each of the images are processed by the image processor 1710. The image processor 1710 may be implemented by one of the various models, such as the DCNN discussed in the preceding examples, which may extract spatial feature information from the images of the image corpus 1705. The images of the image corpus 1705 and the spatial features may be added to the database 1740. The database 1740 may be implemented by the historical database 150. The image and position within the image of the spatial features is tracked for later retrieval from the database 1740.

A second indexing step may then be performed once all the data of the image corpus 1705 has been ingested and resolved into spatial features. The second step involves learning a dictionary of features by first clustering all the extracted features and then associating each computed feature with the closest cluster. This approach may significantly reduce the enormous number of features into a small number of cluster centroids (usually between 100,000 to 1,000,000 based on the data and application-at-hand). These centroids, commonly referred to as visual words, enable a search to proceed in a hierarchical fashion, greatly reducing the lookup time required for find high quality matches. This type of image indexing has been shown to perform near real-time matching and retrieval in datasets of millions of images without any additional constraints on labels. To handle multiple magnifications, the data processing unit 160 may extract features from different layers (corresponding to different retinal fields or magnifications). Separate dictionaries may then be learned for each level of magnifications and visual words may be indexed accordingly.

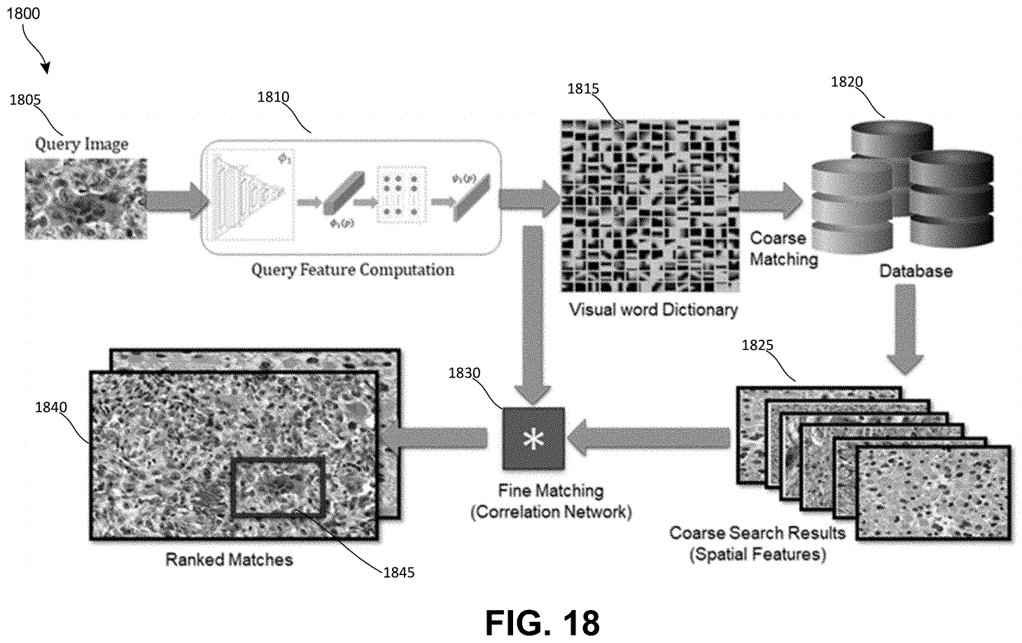

FIG. 18 is a diagram showing an example process 1800 for performing of the query step that may be performed using the personalized oncology system 125. The user may identify a portion or patch of an image 1805 that the user finds interesting and wishes to find similar looking objects and/or textures within the corpus of search data maintained by the historical database 150. The user may identify the portion of the image of interest using the ROI techniques discussed in the preceding examples. The image processing service, which may be implemented by the search unit 145, may be configured to compute a query feature in operation 1810 using a CNN or other machine learning model trained to identify features of the query image 1805. The query feature(s) are computed at appropriate magnification based on the magnification of the query image 1805. Similarly, the magnification of query image 1805 may be used by the search unit 145 to determine which learned dictionary and associated indexes are used in subsequent processing. For efficient retrieval, the search may be performed in two steps: (i) a coarse search step and (ii) a fine search step. In the coarse search step, the query feature obtained in operation 1810 is converted into a visual word by comparing the query feature to each cluster centroid (the visual words in the dictionary 1815) and assigning the closest visual word to the query image 1805. In the fine search step, the candidates from the coarse search 1825 are densely ranked according to their similarity to the query using a correlation network in operation 1830, which may be the similarity network 1400 shown in FIG. 14. The ranked results may then be presented to the user on a user interface provided by the user interface unit 135. The ranked results may be presented with the source image 1840 and the position within the image 1845 from which the ranked results is found. Relevant metadata associated with the source image 1840 and/or the position within the image 1845 may also be presented to the user.

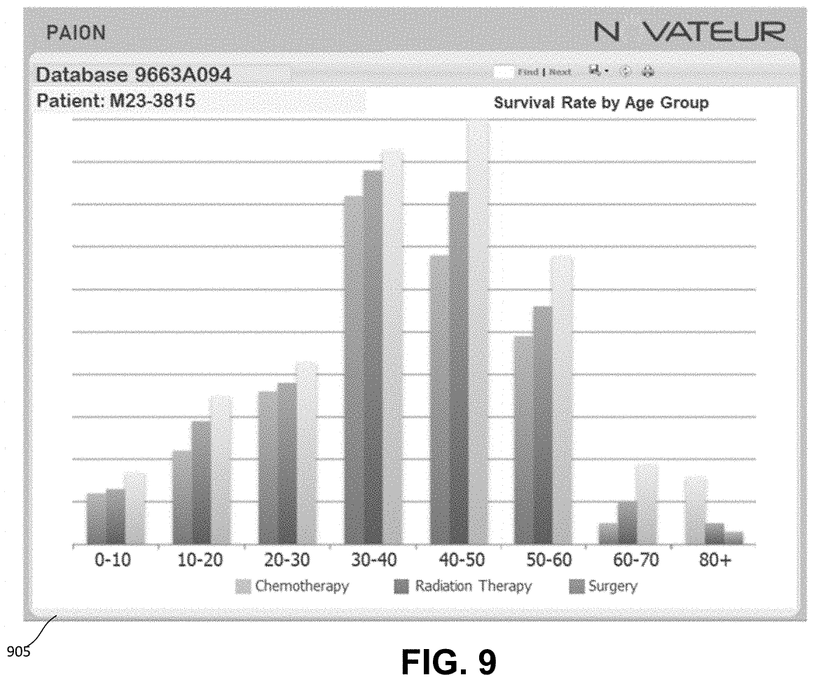

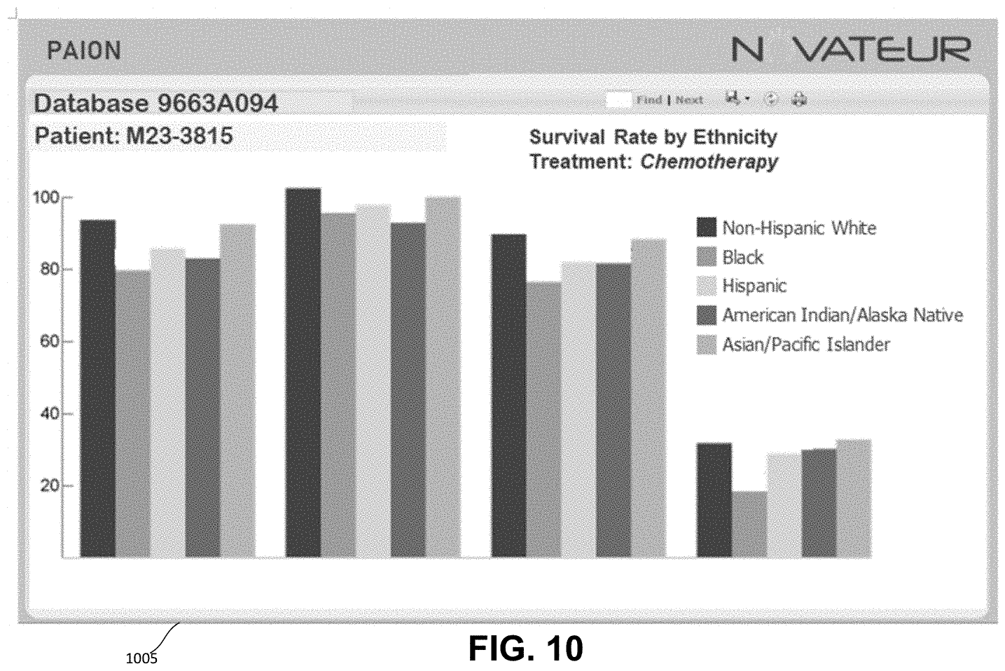

The interaction model and the user interface (UI) components of the personalized oncology system 125 enables pathologists and other users to explore questions, capture answers and understand the legacy and confidence levels for artificial intelligence-based image retrieval system. The web-based UI will provide users with a robust set of tools to query the system and retrieve actionable metrics. Pathologists will use the UI to view the matched image regions and associated clinical data and obtain associated statistics and metrics about mortality, morbidity, and time-to-event (FIGS. 6 and 8). As discussed in the preceding examples, the pathologists will be able to filter the results based on a number of clinical parameters (age, gender, race, co-morbidity, treatment plans, etc.). For a given image sample, the pathologist may be able to see graphical information on various statistics, for example, survival rates of matched patients based on, age-group (FIG. 9) broken-down by treatment type, survival rates by ethnicity for a particular treatment option (FIG. 10), average duration of treatment (for different treatment options), reoccurrence rate and average time-to-reoccurrence, and so on.

The user interface unit 135 of the personalized oncology system 125 may be configured to provide intuitive user interfaces for pathologists and other users to view the matched image regions and associated clinical data, filter the results based on a number of clinical parameters, such as but not limited to age, gender, and treatment plans, and obtain associated statistics and metrics about mortality, morbidity, and time-to-event. FIGS. 8, 9, and 10 show examples of user interfaces that may be implemented by the personalized oncology system 125 to provide pathologists key clinical insights (such as survival rate) based on a number of parameters that include patient's age, gender, race, co-morbidity, and treatment plans.

FIG. 9 is an example user interface 905 of the personalized oncology system 125 for displaying results obtained by searching the historical database 150. The user interface 905 may be implemented by the user interface unit 135 of the personalized oncology system 125. The user interface 905 that may be used to display additional details of the survival rate by age group. The user interface 905 may be displayed in response to the user clicking on the “expand” button shown on the survival rate information 815 section of the user interface 805 shown in FIG. 8. The user interface 905 breaks the survivor rate information into eight age groups: 0-10, 10-20, 20-30, 30-40, 40-50, 50-60, 60-70, and 80+ and provides survivor rate information for each of the three treatment options selected in the user interface 805 of FIG. 8: chemotherapy, surgery, and radiation treatment.

FIG. 10 is an example user interface 1005 of the personalized oncology system 125 for displaying results obtained by searching the historical database 150. The user interface 1005 may be implemented by the user interface unit 135 of the personalized oncology system 125. The user interface 1005 that provides a chart showing the survivor rate information broken down by ethnicity for the chemotherapy treatment. The user interface 1005 may be displayed in response to the user clicking on the “expand” button shown on the survival rate information 815 section of the user interface 805 shown in FIG. 8.

The user interfaces 905 and 1005 are examples of user interfaces that the personalized oncology system 125 may provide to present patient information and historical information to the pathologist to aid the pathologist in developing a personalized therapeutic plan for the patient. The user interfaces provided by the personalized oncology system 125 are not limited to these examples, and other user interfaces that present detailed reports based on the patient data and the historical data may be included. Additional reports may be automatically generated based on the search parameter 810 selected on the user interface 805 of FIG. 8, and the user interface 805 may provide a means for the pathologist to access these additional reports. Furthermore, the personalized oncology system 125 may automatically reports for various permutations of the search parameters 810 to provide the pathologist with reports that may assist the pathologist in developing a personalized therapeutic plan for the patient. For example, a first report may include survival rate information grouped by age and ethnicity and a second report may include survival rate information grouped by age and comorbidity.

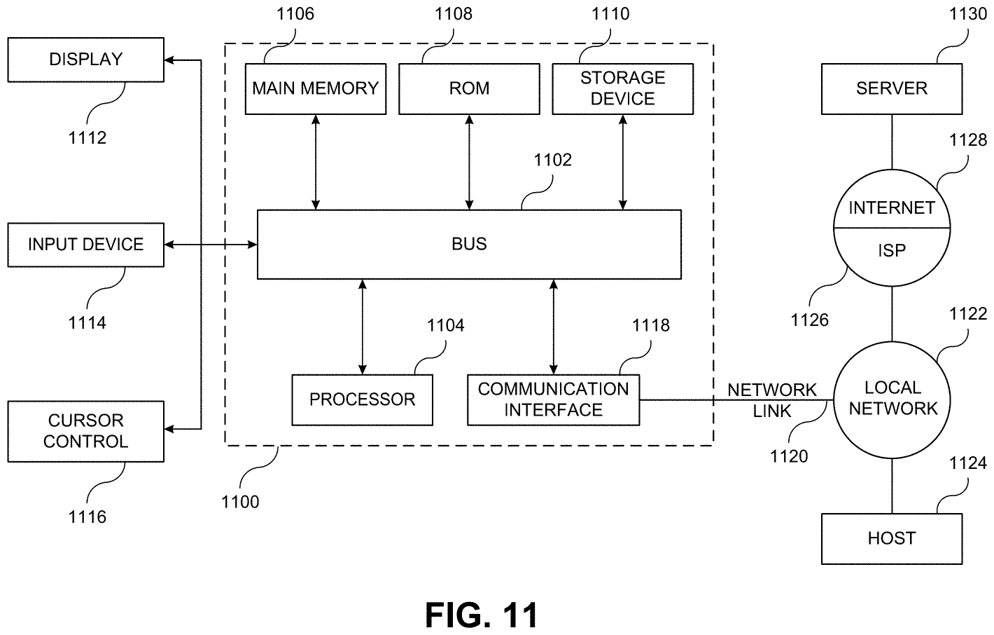

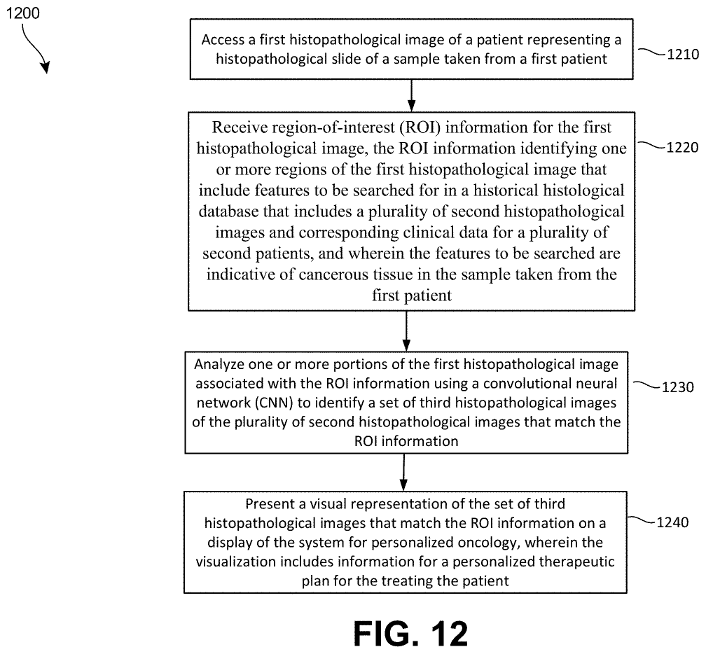

FIG. 12 is a flow chart of an example process 1200 for generating a personalized therapeutic plan for a patient requiring oncological treatment. The process 1200 may be implemented by the personalized oncology system 125. The process 1200 may be implemented on the computing system 1100 shown in FIG. 11.

The process 1200 may include an operation of 1210 of accessing a first histopathological image of a histopathological slide of a sample taken from a first patient. The whole-slide image may be accessed from the pathology database 110 as discussed in the preceding examples. The slide may be accessed by a user via a user interface similar to the user interface 805 shown in FIG. 8.

The process 1200 may include an operation of 1220 of receiving region-of-interest (ROI) information for the first histopathological image. The ROI information identifies one or more regions of the first histopathological image that include features to be searched for in a historical histological database that includes a plurality of second histopathological images and corresponding clinical data for a plurality of second patients. The features to be searched are indicative of cancerous tissue in the sample taken from the first patient. The ROI information may be received via a user via user interface, such as that shown in FIG. 6, and/or the ROI information may be automatically determined by the ROI selection unit 140.

The process 1200 may include an operation of 1230 of analyzing one or more portions of the first histopathological image associated with the ROI information using a convolutional neural network (CNN) to identify a set of third histopathological images of the plurality of second histopathological images that match the ROI information. As discussed in the preceding examples, the portion or portions of the first histopathological image associated with the ROI information may be provided to the CNN as an input. The remainder of the image may be discarded. This can significantly improve the ability of the CNN to match the image data associated with the ROI without requiring large amounts of annotated training data to train the machine models.

The process 1200 may include an operation of 1240 of presenting a visual representation of the set of third histopathological images that match the ROI information on a display of the system for personalized oncology. As discussed with respect to the preceding examples, the visualization includes information for a personalized therapeutic plan for the treating the patient. The visualization information may be rendered on a display of computer system on a user interface like those shown in FIGS. 8-10.

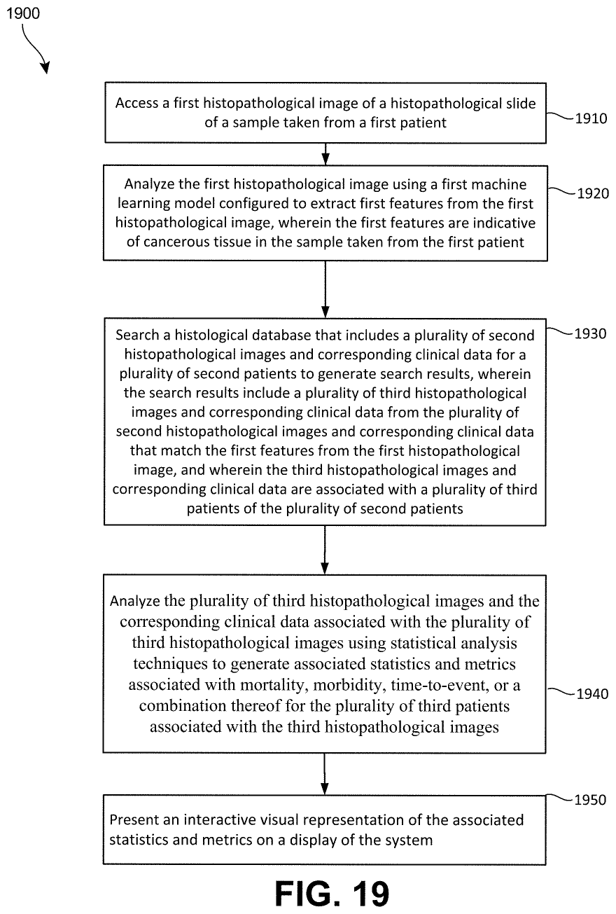

FIG. 19 is a flow chart of an example process 1900 for generating a personalized therapeutic plan for a patient requiring oncological treatment. The process 1900 may be implemented by the personalized oncology system 125. The process 1900 may be implemented on the computing system 1100 shown in FIG. 11. The process may be implemented by the search unit 145 and/or other components of the personalized oncology system 125 discussed in the preceding examples.

The process 1900 may include an operation 1910 of accessing a first histopathological image of a histopathological slide of a sample taken from a first patient. The whole-slide image may be accessed from the pathology database 110 as discussed in the preceding examples. The slide may be accessed by a user via a user interface like the user interface 805 shown in FIG. 8.

The process 1900 may include an operation 1920 of analyzing the first histopathological image using a first machine learning model configured to extract first features from the first histopathological image. The first features may be indicative of cancerous tissue in the sample taken from the first patient. The operation 1920 may be performed by the search unit 145 of the personalized oncology system 125. The first machine learning model may be a DCNN as described with respect to FIGS. 6 and 18.

The process 1900 may include an operation 1930 of searching a histological database that includes a plurality of second histopathological images and corresponding clinical data for a plurality of second patients to generate search results. The search results may include a plurality of third histopathological images and corresponding clinical data from the plurality of second histopathological images and corresponding clinical data that match the first features from the first histopathological image. The third histopathological images and corresponding clinical data are associated with a plurality of third patients that are a subset of the plurality of second patients. This operation may match a subset of the histological images of the historical database 150 to match histopathological images that exhibit the same or similar histology of the first patient. The matching techniques disclosed herein may provide a much larger number of close matches (e.g. ten, hundreds, thousands, or more) than would be otherwise be possible with current approaches to finding matching slides. The current approaches may return one slide or a small number of slides, which is not useful for statistical analysis and predictions that may be used to guide a user in developing a therapeutic plan for the first patient.

The quality of the matches obtained in the operation 1930 may be improved or further refined through the use of genomics data. The historical database 150 may include genomics data associated with the histopathological image data stored therein. The search unit 145 of the personalized oncology system 125 may be configured to analyze the first genomic information obtained from the first patient and to search the historical database 150 for second patients that have similar genomic information that may influence the treatments provided and/or the predicted outcomes of such treatments for the first patient. The search unit 145 may utilize a machine learning model trained to receive genomic information for a patient as an input and/or features extracted therefrom by a feature extraction preprocessing operation. The model may be configured to analyze the genomic information for the second patients included in the historical database 150 and to identify patients having similar features in their genomic data that may influence the treatment plans provided to the first patient and/or the predicted outcomes of such treatments for the first patient. In some implementations, the search unit 145 may be configured to narrow down the search results and/or to rank the search results obtained in operation 1930 that match based on the histology of the first patient and the second patients by using the genomic information identify the search results that may be most relevant to the first patient.

The process 1900 may include an operation 1940 of analyzing the plurality of third histopathological images and the corresponding clinical data associated with the plurality of third histopathological images using statistical analysis techniques to generate associated statistics and metrics associated with mortality, morbidity, time-to-event, or a combination thereof. The associated statistics and metrics may include information for a plurality of subgroups of the plurality of third patients where each respective patient of a subgroup of the plurality of third patients shares one or more common factors with other patients within the subgroup. The common factors may include but are not limited to age, gender, comorbidity, treatments received, and/or other factors that may be indicative of and/or influence the survival rate, the treatment options, and/or other issues associated of the patients having those factors. The personalized oncology system 125 provides this statistical analysis of the histological data from the historical database 150 for patients having a similar histology as the first patient in order to provide informative and accurate information that may predict the survival rate of first patient. The data may be grouped by one or more of these common factors to provide information that predicts a common factor such as age or treatment plan may impact the prognosis and/or the recommended treatment plan for the first patient. Other combinations of common factors may also be determined in addition to or instead of the preceding example in order to provide the user with data that may be used to predict how these combinations of factors may impact the prognosis of the first patient and/or the recommended treatment plan.

The process 1900 may include an operation 1950 of presenting an interactive visual representation of the associated statistics and metrics on a display of the system. The interactive visual representation of the associated statistics and metrics may include interactive reports that allow the user to select one or more common factors that influence survival rate and to obtain survival rate information for the subgroup of third patients that share the one or more common factors with the first patient. The user may interact with the interactive visual representation to develop a therapeutic plan that is tailored to the specific needs of the first patient which may include (i) avoiding unnecessary treatment that is less likely to work for the given patient, (ii) avoiding side effects, trauma, and risks or surgery, and (iii) determining the optimal therapeutic schedules that are best suited for the first patient.

The personalized oncology system 125 may automatically generate a treatment plan for the first patient based on common factors of the first patient and the plurality of third patients. The treatment plan may include recommended treatments for the first patient and information indicating why each of the recommended treatments were recommended for the first patient. The treatment plan may include the information indicating why a particular treatment was selected so that the first patient and the doctor or doctors treating the first patient have a clear understanding of why the recommendations were made. This approach in addition to the “glass box” nature of the models used to provide the recommendations can help to assure the first patient and the doctors that the recommendations are based on data that is relevant to the first user. The personalized oncology system 125 provides the doctors with all the supporting evidence (in the form of historical imagery, matched regions-of-interest, associated clinical data, and genomic data if available) so that the doctors can make confident predictions and clinical decisions.

FIG. 11 is a block diagram showing an example a computer system 1100 upon which aspects of this disclosure may be implemented. The computer system 1100 may include a bus 1102 or other communication mechanism for communicating information, and a processor 1104 coupled with the bus 1102 for processing information. The computer system 1100 may also include a main memory 1106, such as a random-access memory (RAM) or other dynamic storage device, coupled to the bus 1102 for storing information and instructions to be executed by the processor 1104. The main memory 1106 may also be used for storing temporary variables or other intermediate information during execution of instructions to be executed by the processor 1104. The computer system 1100 may implement, for example, the AI-based personalized oncology system 125.

The computer system 1100 may further include a read only memory (ROM) 1108 or other static storage device coupled to the bus 1102 for storing static information and instructions for the processor 1104. A storage device 1110, such as a flash or other non-volatile memory may be coupled to the bus 1102 for storing information and instructions.

The computer system 1100 may be coupled via the bus 1102 to a display 1112, such as a liquid crystal display (LCD), for displaying information. One or more user input devices, such as the example user input device 1114 may be coupled to the bus 1102, and may be configured for receiving various user inputs, such as user command selections and communicating these to the processor 1104, or to the main memory 1106. The user input device 1114 may include physical structure, or virtual implementation, or both, providing user input modes or options, for controlling, for example, a cursor, visible to a user through display 1112 or through other techniques, and such modes or operations may include, for example virtual mouse, trackball, or cursor direction keys. Some implementations may include a cursor control 1116 which is separate from the user input device 1114 for controlling the cursor. In such implementations, the user input device 1114 may be configured to provide other input options, while the cursor control 1116 controls the movement of the cursor. The cursor control 1116 may be a mouse, trackball, or other such physical device for controlling the cursor.

The computer system 1100 may include respective resources of the processor 1104 executing, in an overlapping or interleaved manner, respective program instructions. Instructions may be read into the main memory 1106 from another machine-readable medium, such as the storage device 1110. In some examples, hard-wired circuitry may be used in place of or in combination with software instructions. The term “machine-readable medium” as used herein refers to any medium that participates in providing data that causes a machine to operate in a specific fashion. Such a medium may take forms, including but not limited to, non-volatile media, volatile media, and transmission media. Non-volatile media may include, for example, optical or magnetic disks, such as storage device 1110. Transmission media may include optical paths, or electrical or acoustic signal propagation paths, and may include acoustic or light waves, such as those generated during radio-wave and infra-red data communications, that are capable of carrying instructions detectable by a physical mechanism for input to a machine.

The computer system 1100 may also include a communication interface 1118 coupled to the bus 1102, for two-way data communication coupling to a network link 1120 connected to a local network 1122. The network link 1120 may provide data communication through one or more networks to other data devices. For example, the network link 1120 may provide a connection through the local network 1122 to a host computer 1124 or to data equipment operated by an Internet Service Provider (ISP) 1126 to access through the Internet 1128 a server 1130, for example, to obtain code for an application program.

While various embodiments have been described, the description is intended to be exemplary, rather than limiting, and it is understood that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached summary statements and their equivalents. Also, various modifications and changes may be made within the scope of the attached summary statements.

What is claimed is:

1. A system for personalized oncology, comprising:
a processor; and
a memory in communication with the processor, the memory comprising executable instructions that, when executed by the processor, cause the processor to control the system to perform functions of:
accessing a first histopathological image of a histopathological slide of a sample taken from a first patient;
analyzing the first histopathological image using a first machine learning model configured to extract first features from the first histopathological image, wherein the first features are indicative of cancerous tissue in the sample taken from the first patient;
searching a histological database that includes a plurality of second histopathological images and corresponding clinical data for a plurality of second patients to generate search results, wherein the search results include a plurality of third histopathological images and corresponding clinical data from the plurality of second histopathological images and corresponding clinical data that match the first features from the first histopathological image, and wherein the third histopathological images and corresponding clinical data are associated with a plurality of third patients of the plurality of second patients;
analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and
presenting an interactive visual representation of the associated statistics and metrics on a display of the system.

2. The system of claim 1, wherein the associated statistics and metrics include associated statistics and metrics for a plurality of subgroups of the plurality of third patients, and wherein each respective patient of a subgroup of the plurality of third patients shares one or more common factors with other patients within the subgroup.

3. The system of claim 1, wherein the one or more common factors include one or more of age, gender, race, comorbidity, genomic profiles, and treatments received.

4. The system of claim 1, further comprising:
analyzing first genomic profile information associated with the first patient; and
matching the first genomic profile information with genomic profile information associated with a subset of the plurality of second patients to generate the search results.

5. The system of claim 1, further comprising instructions configured to cause the processor to control the system to perform functions of:
automatically generating a treatment plan for the first patient based on common factors of the first patient and the plurality of third patients.

6. The system of claim 1, further comprising instructions configured to cause the processor to control the system to perform functions of:
receiving region-of-interest (ROI) information for the first histopathological image, the ROI information identifying one or more regions of the first histopathological image that include features to be searched for in the historical histological database,
wherein to analyze the first histopathological image using the first machine learning model configured to extract the first features from the first histopathological image, the memory further comprising instructions configured to cause the processor to control the system to perform functions of:
analyzing the one or more regions of the first histopathological image associated with the ROI information using the first machine learning model to extract the first features.

7. The system of claim 6, wherein to receive the ROI information for the first histopathological image, the memory further comprising instructions configured to cause the processor to control the system to perform functions of:
displaying the first histopathological image on a first user interface of the system for personalized oncology; and
receiving, via the first user interface, user input defining the ROI information for the one or more regions of the first histopathological image that include features to be searched.

8. The system of claim 6, wherein to receive the ROI information for the first histopathological image, the memory further comprising instructions configured to cause the processor to control the system to perform functions of:
analyzing the first histopathological image using a second machine learning model trained to automatically identify areas of interest in the first histopathological image; and
receiving the ROI information for the one or more regions of the first histopathological image that include features to be searched.

9. The system of claim 8, wherein to analyze the first histopathological image using the second machine learning model trained to automatically identify areas of interest in the first histopathological image further comprising instructions configured to cause the processor to control the system to perform functions of:
automatically identifying the areas of interest based on characteristics including one or more of nuclear atypia, mitotic activity, cellular density, or tissue architecture to identify cancer cells.

10. The system of claim 1, further comprising instructions configured to cause the processor to control the system to perform functions of:
receiving one or more search parameters associated with one or more clinical data elements associated with the first patient;
filtering the set of third histopathological images based on the one or more search parameters and clinical data associated with the third histopathological images to generate a set of fourth histopathological images; and
presenting the interactive visual representation of the set of fourth histopathological image data instead of the third histopathological images.

11. A method of operating a personalized oncology system, the method comprising:
accessing a first histopathological image of a histopathological slide of a sample taken from a first patient;
analyzing the first histopathological image using a first machine learning model configured to extract first features from the first histopathological image, wherein the first features are indicative of cancerous tissue in the sample taken from the first patient;
searching a histological database that includes a plurality of second histopathological images and corresponding clinical data for a plurality of second patients to generate search results, wherein the search results include a plurality of third histopathological images and corresponding clinical data from the plurality of second histopathological images and corresponding clinical data that match the first features from the first histopathological image, and wherein the third histopathological images and corresponding clinical data are associated with a plurality of third patients of the plurality of second patients;
analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and
presenting an interactive visual representation of the associated statistics and metrics on a display of the system.

12. The method of claim 11, wherein the associated statistics and metrics include associated statistics and metrics for a plurality of subgroups of the plurality of third patients, and wherein each respective patient of a subgroup of the plurality of third patients shares one or more common factors with other patients within the subgroup, and wherein the one or more common factors include one or more of age, gender, race, comorbidity, and treatments received.

13. The system of claim 11, further comprising:
receiving region-of-interest (ROI) information for the first histopathological image, the ROI information identifying one or more regions of the first histopathological image that include features to be searched for in the historical histological database,
wherein analyzing the first histopathological image using the first machine learning model configured to extract the first features from the first histopathological image further comprises:
analyzing the one or more regions of the first histopathological image associated with the ROI information using the first machine learning model to extract the first features.

14. The method of claim 13, wherein the first machine learning model is configured to perform feature extraction on the one or more regions of the first histopathological image to generate extracted features and to compare the extracted features to features of the plurality of second histopathological images.

15. The method of claim 14, wherein receiving the ROI information for the first histopathological image further comprises:
displaying the first histopathological image on a first user interface of the system for personalized oncology; and
receiving, via the first user interface, user input defining the ROI information for the one or more regions of the first histopathological image that include features to be searched.

16. The method of claim 14, wherein receiving the ROI information for the first histopathological image further comprises:
analyzing the first histopathological image using a second machine learning model trained to automatically identify areas of interest in the first histopathological image; and
receiving the ROI information for the one or more regions of the first histopathological image that include features to be searched.

17. The method of claim 16, wherein analyzing the first histopathological image using the second machine learning model trained to automatically identify areas of interest in the first histopathological image further comprises:
automatically identifying the areas of interest based on characteristics including one or more of nuclear atypia, mitotic activity, cellular density, or tissue architecture to identify cancer cells.

18. The method of claim 14, further comprising:
receiving one or more search parameters associated with one or more clinical data elements associated with the first patient;
filtering the set of third histopathological images based on the one or more search parameters and clinical data associated with the third histopathological images to generate a set of fourth histopathological images; and
presenting the interactive visual representation of the set of fourth histopathological image data instead of the third histopathological images.

19. A non-transitory computer readable medium containing instructions which, when executed by a processor, cause a computer to perform functions of:
accessing a first histopathological image of a histopathological slide of a sample taken from a first patient;
analyzing the first histopathological image using a first machine learning model configured to extract first features from the first histopathological image, wherein the first features are indicative of cancerous tissue in the sample taken from the first patient;
searching a histological database that includes a plurality of second histopathological images and corresponding clinical data for a plurality of second patients to generate search results, wherein the search results include a plurality of third histopathological images and corresponding clinical data from the plurality of second histopathological images and corresponding clinical data that match the first features from the first histopathological image, and wherein the third histopathological images and corresponding clinical data are associated with a plurality of third patients of the plurality of second patients;
analyzing the plurality of third histopathological images and the corresponding clinical data associated with the plurality of third histopathological images using statistical analysis techniques to generate associated statistics and metrics associated with mortality, morbidity, time-to-event, or a combination thereof for the plurality of third patients associated with the third histopathological images; and
presenting an interactive visual representation of the associated statistics and metrics on a display of the system.

20. The non-transitory computer readable medium of claim 19, wherein the associated statistics and metrics include associated statistics and metrics for a plurality of subgroups of the plurality of third patients, and wherein each respective patient of a subgroup of the plurality of third patients shares one or more common factors with other patients within the subgroup, and wherein the one or more common factors include one or more of age, gender, race, comorbidity, and treatments received.
